(19) United States
(12) Patent Application Publication
(10) Pub. No.: US 2022/0062391 A1
(43) Pub. Date: Mar. 3, 2022

(54) ENHANCING EPITHELIAL OR ENDOTHELIAL BARRIER FUNCTION

(71) Applicant: UNIVERSITY OF ROCHESTER, Rochester, NY (US)

(72) Inventor: David Dean, Rochester, NY (US)

(21) Appl. No.: 17/420,732

(22) PCT Filed: Jan. 15, 2020
(86) PCT No.: PCT/US2020/013599

§ 371 Date: Jul. 6, 2021

Related U.S. Application Data
(60) Provisional application No. 62/793,088, filed on Jan. 16, 2019.

(51) Int. Cl. A61K 38/45; A61K 38/46; A61P 11/00

(52) U.S. Cl. CPC A61K 38/45; C12Y 207/11001; A61P 11/00; C12Y 306/01; A61K 38/46

(57) ABSTRACT

The present invention relates to improvement of epithelial or endothelial barrier function by increasing a level of myotonic dystrophy kinase-related Cdc42-binding kinases a (MRCKalpha) in one or more cells in the barrier.

### CROSS REFERENCE TO RELATED APPLICATION

This application claims priority to U.S. Provisional Application No. 62/793,088 filed on Jan. 16, 2019. The content of the application is incorporated herein by reference in its entirety.

### GOVERNMENT INTERESTS

This invention was made with government support under HL120521 and HL131143 awarded by the National Institutes of Health. The government has certain rights in the invention.

### FIELD OF THE INVENTION

This invention relates to improvement of epithelial or endothelial barrier function.

### BACKGROUND OF THE INVENTION

Epithelial and endothelial barriers are essential to life. While the endothelium lines the vasculature and ensures tissue supply with nutrients and oxygen, the epithelium forms the barrier between tissues and the outer environment thus protecting organs from invading harmful agents. Both barriers also play a critical role in the innate immune response to injury and infection. Accordingly, dysfunction in epithelial or endothelial barrier underlies various diseases. For example, acute respiratory distress syndrome (ARDS) is a life-threatening lung condition that affects over 190,600 people each year in the United States and accounts for 74,500 deaths (1, 2). The injury of the alveolar epithelial and endothelial barriers is the hallmark of ARDS, which can be induced by several factors, including infection, aspiration syndromes, blood transfusion, and mechanical forces (4). While the damage and repair of the endothelial barrier is well-characterized, the mechanism of epithelial injury and repair is poorly understood. Current therapy relies on supportive care, rather than targeting the underlying pathophysiology of the disease (3). Thus, there is a need for agents and methods for improving epithelial and endothelial barrier function.

### SUMMARY OF INVENTION

This invention addresses the need mentioned above in a number of aspects.

In one aspect, the invention features a method of improving integrity or function of an epithelial or endothelial barrier. The method comprises increasing a level of myotonic dystrophy kinase-related Cdc42-binding kinases α (MRCKα) in one or more cells in the barrier. In some examples, the barrier is an epithelial barrier, such as an alveolar epithelial barrier. The level of MRCKα can be an enzymatic level or an expression level of an MRCKα gene. Increasing the level of MRCKα can comprise introducing an MRCKα polypeptide or a first nucleic acid encoding the MRCKα polypeptide into the one or more cells. The method can further comprise increasing a level of Nat, -ATPase (NKA) β1 subunit (e.g., an activity level or an expression level of NKA gene β1) in the one or more cells. This can be achieved by increasing the level of the NKA β1 subunit polypeptide by introducing an NKA β1 subunit polypeptide or a second nucleic acid encoding the NKA β1 subunit polypeptide into the cells. Furthermore, the level of NKA β1 subunit may be an activity level or an expression level of NKA β1 gene. The cells can be alveolar epithelial cells, which can be in vitro or in vivo in a subject. The first nucleic acid or the second nucleic acid can be in a same expression vector or two different expression vectors.

Also provided is a method of treating a disease or condition associated with compromised function of an epithelial or endothelial barrier. The method comprises increasing a level of MRCKα in one or more cells in the epithelial or endothelial barrier of a subject in need thereof. Examples of the disease or condition includes one selected from the group consisting of acute lung injury, acute respiratory distress syndrome (ARDS), and asthma.

In a second aspect, the invention provides a nucleic acid molecule or a set of nucleic acid molecules encoding the MRCKα and NKA β1 subunit mentioned above. The nucleic acid molecule or the set of nucleic acid molecules can be isolated or present in an expression cassette, a vector, a host cell, a virus or a virus-like particle.

The invention further provides a pharmaceutical composition comprising (i) the nucleic acid molecule or the set of nucleic acid molecules, the vector, the host cell, the virus or virus-like particle described above and (ii) a pharmaceutically acceptable carrier or excipient. Also provided is a kit comprising one or more of the nucleic acid molecule, the set of nucleic acid molecules, the vector, the host cell, the virus, and the virus-like particle.

The details of one or more embodiments of the invention are set forth in the description below. Other features, objectives, and advantages of the invention will be apparent from the description and from the claims.

### BRIEF DESCRIPTION OF THE DRAWINGS

FIGS. 1A, 1B, 1C, 1D, and 1E are a set of diagrams and photographs showing that overexpression of NKA β1 (β1) subunit increased alveolar type I barrier integrity. (FIG. 1A) Rat primary ATII cells differentiated into phenotypic ATI cells when cultured in vitro. Cells were lysed for qPCR analysis of ATII marker (SPC) and ATI marker (T1a). (FIG. 1B) Cells were electroporated with plasmid expressing the rat β1 subunit or the pCDNA3 empty plasmid as control at day 3 post isolation. Cells were lysed for western blot 24 hours later. (FIG. 1C) Quantification of the western blots for ATI cells. (FIG. 1D) ATII cells were isolated and transfected immediately with plasmids expressing the rat β1 subunit or the pCDNA3 empty plasmid as control. Cells were lysed for western blot 24 hours later. (FIG. 1E) Quantification of the western blots for ATII cells. *p<0.05, **p<0.01.

FIGS. 2A, 2B, and 2C are a set of diagrams and photographs showing that overexpression of β1 subunit increased alveolar type I barrier function. (FIG. 2A) ATI cells (day 3 after isolation) were electroporated with plasmid expressing the rat β1 subunit or the pCDNA3 empty plasmid as control. 24 hours later, cells were stained for occludin, zo-1, zo-2 and claudin-4. Red shows staining of tight junction proteins and blue shows DAPI for nuclear staining. Scale bar: 70 mm (FIG. 2B) ATII cells were isolated, then cotransfected with 4 mg/ml pCMV-Tet3G plasmid and 16 mg/ml pTet3G-human β1 plasmid day 1 after isolation. Cells were then plated on fibronectin-coated 12-well transwell plates. 24 hours later at day 2, 1 μg/ml of dox were added to induce β1 gene expression. TEER was measured every 24 hours. Two-way ANOVA was used for statistical analysis. ***p<0.001. (FIG. 2C) After TEER measurement at day 4, permeability to 3 kD Texas red-dextran and 401(D FITC-dextran was measured for a duration of 2 hours. **p<0.01.

FIGS. 3A, 3B, and 3C are a set of photographs showing that β1 subunit mediated tight junction upregulation is ion-transport independent. (FIG. 3A) ATI cells (day 3 after isolation) were transfected with plasmid expressing the mouse β2 subunit or the pCDNA3 empty plasmid as control. Cells were lysed for western blot analysis after 24 hours. (FIG. 3B) ATI cells (day 3 after isolation) were transfected with plasmid expressing the mouse β3 subunit containing a DDK tag or the pCDNA3 empty plasmid as control. Cells were lysed for western blot analysis after 24 hours. (FIG. 3C) ATI cells were transfected with plasmid expressing the rat β1 subunit or the pCDNA3 empty plasmid, then immediately treated with ouabain at 0, 10 nM, 100 nM or 1000 nM. Twenty-four hours later, western blot was performed.

FIGS. 4A, 4B, 4C, and 4D are a set of photographs and diagram showing that MRCKα interacted with the β1 subunit and stabilizes tight junctions. (FIG. 4A) The interaction of MRCKα with the β1 subunit was confirmed using by co-immunoprecipitation. 5% of total cell lysate was used for input. The β2 or β3 subunit did not co-immunoprecipitate with MRCKα. (FIG. 4B) β1 subunit and MRCKα co-stains in ATI cells. Scale bar: 20 μm. (FIG. 4C) ATI cells were transfected with a scrambled siRNA (siScramble) or a siRNA against MRCKα (siMRCKα). Twenty-four hours later, cells were lysed for immunoblot analysis. (FIG. 4D) Densitometry of the western blot in (C).

FIGS. 5A, 5B, and 5C are a set of diagrams and photographs showing that MRCKα was required for β1-mediated alveolar barrier tightening. (FIG. 5A) ATII cells were cotransfected with siRNA (scramble control or against MRCKα) and plasmids (CMV-tet and Tet-β1) at 24 hours after isolation. 1 μg/μl dox was added to induce gene expression at day 2. TEER was then measured every 24 hours from day 3 to day 5. (FIG. 5B) ATII cells were cotransfected with plasmids (CMV-tet and Tet-β1) at 24 hours after isolation and treated immediately with 2 μM of MRCKα inhibitor BDP5290. TEER was measured 24 hours later. Statistical analysis was performed using Two-way ANOVA. *p<0.05, **p<0.01, ***p<0.001. (FIG. 5C) Immunofluorescence staining of zo-1 in cells treated with or without doxcyline for 48 hours after transfected with luciferase, β1 and siScramble, or β1 and siMRCKα. Images represent three separate experiments. Scale bar: 100 um.

FIGS. 6A and 6B are a diagram and a set of photographs showing that overexpression of MRCKα increased epithelial barrier function. (FIG. 6A) ATII cells were transfected with empty plasmid or MRCKα one day after isolation. TEER was then measured at a 24-hour interval. Statistical analysis was performed using Two-way ANOVA. N=6, ****p<0.0001. (FIG. 6B) Immunofluorescence staining of occludin and zo-1 48 hours after transfection. Data are representative of three independent experiments. Scale bar: 70 um.

FIGS. 7A, 7B, and 7C are a diagram and a set of photographs showing that the MRCKα downstream pathway was activated upon overexpression of the β1 subunit. (FIG. 7A) Cells were electroporated with plasmid expressing the rat β1 subunit or the pCDNA3 empty plasmid as control at day 3 post isolation. Cells were lysed for western blot 24 hours later. (FIG. 7B) At day 1 after isolation, cells were cotransfected with pCMV-tet and pTet-β1 and treated with 20 μM of blebbistatin or DMSO as control. After another 24 hours, 1 μg/ml of doxycycline was added to induce gene expression. TEER was measured after 24 hours. ***p<0.001. (FIG. 7C) ATI cells were transfected with plasmid expressing the rat β1 subunit or the pCDNA3 empty plasmid, then immediately treated with blebbistatin at a final concentration of 0, 1 uM, 10 uM or 100 uM. Western blot for occludin was performed 24 hours later.

FIGS. 8A, 8B, and 8C are a set of photographs and diagrams showing decreased MRCKα level in the alveolar epithelium of human ARDS patients. (FIG. 8A) Representative images of immunofluorescence of lung sections for MRCKα (green) of a control donor and a patient of ARDS. Upper panel shows images taken at 20× objective magnification and lower panel shows images taken at 63×objective magnification for the boxed region in the upper panel. (FIG. 8B) Quantification of MRCKα expression in the alveoli. ROI (region of interest) were drawn in the alveoli region, and the ratio of integrated pixel intensity for MRCK and DAPI was calculated for each ROI. A total of three normal donors and five ARDS patients were used for quantification, with three random fields were chosen for each sample. Data are expressed as mean±SD, N=9 for normal control and N=15 for ARDS, two-tailed t test, ****p<0.0001. (FIG. 8C) Working model of the β1 subunit increases alveolar epithelial barrier integrity. The β1 subunit of the Na+, K+-ATPase interacts with MRCKα, assists in its activation, leads to higher myosin phosphorylation, and eventually stabilizes tight junctions.

FIGS. 9A, 9B, 9C, and 9D are a set of diagrams and photographs showing characterizing of differentiating of ATII cells to ATI cells. (FIG. 9A) ATI cells were cultured on a 12-well transwell plate with 20 mg/ml of fibronectin coating or PBS as control. TEER is measured every 24 hours after plating (n=4 for each group). (FIG. 9B) After day 4, cells were fixed and stained for occludin and zo-1. Images were taken at 20×, red: occludin or zo-1; blue: DAPI. (FIG. 9C) At day 3 after isolation when cells became confluent and displayed ATI phenotype, 1 μg/ml of LPS was added and TEER was measured 24 hours later, subsequently, (FIG. 9D) permeability to 3 kD FITC-dextran was assayed. *p<0.05; **p<0.01.

FIGS. 10A and 10B are a set of photographs and diagram showing: (FIG. 10A) ATI cells (day 3 after isolation) were electroporated with GFP plasmid using a square wave of 300 V and 20 milliseconds. 24 hours after transfection, cells were imaged for phase contrast and GFP in the same field. Three representative images were showing. Scale bar: 70 mm (FIG. 10B) At day 3 after ATII isolation, cells were electroporated with plasmid expressing the rat β1 subunit or the pCDNA3 empty plasmid as control. mRNA were collected for quantitative PCR analysis 24 hours after electroporation.

FIGS. 11A and 11B are a set of photographs and diagram showing: (FIG. 11A) 16HBE14o-cells were cotransfected with pCMV-tet regulator plasmids and pTet3G-human β1 subunit expressing plasmids by electroporation, followed immediately by addition of 0, 1, 10, 100, and 1000 ng/ml of doxycycline. Cells were lysed for western blot analysis 24 hours after electroporation. (FIG. 11B) 16HBE14o-cells were cotransfected with pCMV-tet plasmids and tet-lucifersase plasmids by electroporation. After transfection, cells were treated with 1 μg/ml of doxycycline or H₂O as control. Cells were lysed with reporter lysis buffer every other day and luminescence was measured. RLU: relative luminescence unit.

FIG. 12 shows sequence fragments of MRCKα (SEQ ID NO: 2) that were detected in mass spectrometry (underlined).

FIGS. 13A, 13B, and 13C are a set of photographs showing decreased MRCKα level in lung sections from human ARDS patients. (FIG. 13A). Staining of MRCKα from sections of 3 normal control donors and 6 ARDS patients. Three random fields were chosen for each patient for intensity quantitation Images from ARDS patient #5 were excluded from analysis due to high background signal. (FIG. 13B). Co-staining of MRCKα and occludin in small way from control donor. (FIG. 13C) Representative staining of MRCKα in airway of control donor and ARDS patient.

FIGS. 14A and 14B are a set of diagrams showing the role of MRCKα in vivo. Male C57black6 mice (n=6) were injured with LPS (5 mg/kg) by intratracheal administration and one day later DNA (100 ug in 50 ul PBS) was delivered to the lungs by transthoracic electroporation, as indicated. Mice received plasmids expressing no protein (pcDNA3), the Na,K-ATPase β1 subunit, MRCKα, or a combination of Na,K-ATPase β1 subunit and MRCKα plasmids. A set of naive mice were also used that received no LPS and no DNA. Two days after DNA delivery, lungs were removed for (FIG. 14A) graviometric analysis of pulmonary edema (wet to dry ratio) and (FIG. 14B) collection of bronchoalveolar lavage fluid (BAL) for measurement of infiltrating immune cells in the airspace. Statistical analysis was by two-way ANOVA and p-values are indicated in the figure.

FIG. 15 is a table showing top 15 interacting proteins of the β1 subunit.

FIG. 16 is a table showing known β1-interacting proteins from the literature.

FIG. 17 is a diagram showing that treatment of LPS-injured lungs with MRCKα reduced pulmonary edema. LPS (5 mg/kg) was administered by aspiration to mice and 1 day later, 100 μg of plasmid in 50 μl was delivered to the lungs by electroporation. After 2 days, lungs were removed for gravimetric analysis. Wet-to-dry ratios are shown as mean±SEM (n=11-13). Statistical analysis was by two way ANOVA. *P<0.05 or ***P<0.001 compared to pCDNA3.

FIG. 18 is a set of photographs showing that treatment of LPS-injured lungs with MRCKα reduces lung injury. LPS (5 mg/kg) was administered by aspiration to mice and 1 day later, 100 μg of plasmid in 50 μl was delivered to the lungs by electroporation (n=6). After 2 days, lungs were removed, inflation fixed with formalin, embedded in paraffin and sectioned. Hematoxylin and eosin staining were used to compare the histological features. Representative images are shown.

FIGS. 19A, 19B, and 19C are a set of photographs and diagrams showing that treatment of LPS-injured lungs with MRCKα reduces PMNs in the BALF. LPS (5 mg/kg) was administered by aspiration to mice and 1 day later, 100 μg of plasmid in 50 μl was delivered to the lungs by electroporation (n=6). Two days later, BALF was collected from the mice, cells were counted (FIG. 19B), and differential staining (FIG. 19A) by cytospin was used to quantitate the numbers of infiltrating PMNs (FIG. 19C). Statistical analysis was by two way ANOVA. **P<0.01 or **P<0.001 compared to pCDNA3.

FIG. 20 is a set of photographs showing that treatment of LPS-injured lungs with MRCKα increases levels of tight junction proteins. LPS (5 mg/kg) was administered by aspiration to mice and 1 day later, 100 μg of plasmid in 50 μl or saline alone was delivered to the lungs by electroporation (n=6). Another group of animals received LPS alone without any further administrations or electroporation. Two days later, lungs were removed for western blot analysis of occludin and ZO-1 levels.

FIG. 21 is a diagram showing that treatment of LPS-injured lungs with MRCKα attenuates LPS-induced pulmonary leakage. Mice were treated with intratracheal LPS (5 mg/kg) and one day later, electroporated with the indicated plasmids. Pulmonary permeability was measured by Evan's blue dye (EBD) leakage from blood to airways. EBD (30 mg/kg) was administrated by tail-vein injection 47 hours after gene transfer. One hour later, lungs were perfused to remove EBD in the vasculature. Extracted EBD from the perfused lungs was quantified by measuring at 620 nm and 740 nm and shown as mean±SEM (n=7-11). Statistical analysis was by two way ANOVA.

FIG. 22 is a diagram showing that overexpression of MRCKα had no effect on alveolar fluid clearance in mouse lungs. One hundred μg of plasmid in 50 μl was administered to mouse lungs by aspiration and electroporation. Two days later, AFC was measured in living mice and is shown as percentage of total instilled volume cleared in 30 min Procaterol (10⁻⁸M in the Evans blue dye instillate) was added to one cohort of mice that had received no transgene as a positive control. Statistical analysis was by one way ANOVA (mean±SEM; n=6-7).

### DETAILED DESCRIPTION OF THE INVENTION

This invention is based, at least in part, on an unexpected discovery of a signaling pathway by which the Na⁺, K⁺-ATPase β1 subunit regulates alveolar tight junctions. As both endothelium and epithelium form tight junctions, the invention is useful to improve epithelial or endothelial barrier function and to treat related disorders such as certain lung disorders and pulmonary diseases.

An intact epithelial barrier is indispensable for lung function and homeostasis. Disruption of the barrier can cause severe diseases such as ARDS, a fatal lung condition with up to 40% mortality. The Na⁺, K⁺-ATPase (NKA), an ion pump expressed in all mammalian cells, has been shown to play a critical role in the pathogenies of ARDS by affecting lung barrier integrity. However, the underlying mechanism is unknown. Here with genetic, pharmacological, and tandem mass spectrometry approaches, it was demonstrated that the NKA β1 subunit potentiates the alveolar epithelial tight junctions in a pump-independent mechanism that requires MRCKα, a protein kinase that regulates the actin cytoskeleton. By interacting with MRCKα, the β1 subunit increases myosin light chain activation and stabilizes expression of tight junctions. This effect is specific for the β1 subunit but not the β2 or the β3 isoform. Importantly, the expression of MRCKα in the alveoli and small airways is significantly decreased in ARDS patients. Taken together, the data disclosed herein has elucidated the molecular pathway of alveolar barrier tightening by the NKA β1 subunit, paving the way for developing new therapies for ARDS and other barrier-associated human diseases.

### Alveolar Epithelial/Endothelial Barrier and MRCKα

The alveolar epithelial barrier is composed of alveolar epithelial type I cells (ATI) and alveolar type II cells (ATII). Tight junctions expressed in both cell types orchestrate tissue integrity and limit the free passage of most ions, solutes, and proteins under normal condition, but becomes leaky in diseases such as ARDS (5, 6). Tight junctions are composed of a large family of proteins, including transmembrane proteins (occludin, claudins, and JAM), scaffolding proteins (zo-1, zo-2, zo-3, Cingulin, etc.), and signaling proteins (Rho family GTPase, kinases, phosphatases) (7). Compared to the current knowledge of how tight junctions break down, few pathways have been described to restore its expression and function, especially in the lung epithelial tissues.

In addition to the tight barrier function, the other important property of the alveolar epithelial barrier is the fluid balance. The ion channels and transporters expressed on both ATI and ATII maintain the lung fluid balance through vectoral ion transport across the epithelial barrier. Among them, the Na⁺, K⁺-ATPase is the most important. The Na⁺, K⁺-ATPase is a heterodimer of the catalytic α subunit and the noncatalytic β subunit, which facilitates the maturation and membrane targeting of the α subunit. In ARDS, both subunits can have decreased expression or disrupted targeting to the basolateral membrane, which lead to the development of lung edema (8-11).

Aiming to restore the ion transport and reduce the lung edema, studies have performed to augment its expression via direct gene delivery of the Na⁺, K⁺-ATPase (12-18). Surprisingly, the noncatalytic β1 subunit was found to confer protection to the alveolar epithelial barrier, as demonstrated by increased expression of tight junction proteins and decreased alveolar barrier permeability (13, 18). However, the underlying molecular mechanism is unknown.

As disclosed herein, studies were carried out to elucidate the signaling pathway by which the Na⁺, K⁺-ATPase β1 subunit regulates alveolar tight junctions. It was first demonstrated that the barrier-enhancing effect of the Na⁺, K⁺-ATPase is specific to the β1 subunit and appears to be independent on the ion-transport activity. Using mass spectrometry, many new interacting partners of the β1 subunit were identified.

Among them is MRCKα, a Serine/Threonine protein kinase. Using loss-of-function, chemical inhibition, and gain-of-function experiments, it was revealed that a novel molecular pathway by which the β1 subunit binds and activates MRCKα, thereby phosphorylates non-muscle myosin II and increase tight junction expression. Interestingly, the protein expression of MRCKα is greatly reduced in patients with ARDS. Taken together, this invention has identified a new molecular pathway from the Na⁺, K⁺-ATPase to alveolar epithelial tight junctions. Targeting this pathway provides a new therapeutic strategy to treat ARDS. List below are the cDNA sequence of MRCKα (also known as CDCl42BPA) open reading frame (ORF; SEQ ID NO: 1) and amino acid sequence (SEQ ID NO: 2):

SEQ ID NO: 1:
atgtctggagaagtgcgtttgaggcagttggagcagtttattttggacgggcccgctcagaccaatgggcagtgcttcagtgt
ggagacattactggatatactcatctgcctttatgatgaatgcaataattctccattgagaagagagaagaacattctcgaat
acctagaatgggctaaaccatttacttctaaagtgaaacaaatgcgattacatagagaagactttgaaatattaaaggtgatt
ggtcgaggagcttttggggaggttgctgtagtaaaactaaaaaatgcagataaagtgtttgccatgaaaatattgaataaatg
ggaaatgctgaaaagagctgagacagcatgttttcgtgaagaaagggatgtattagtgaatggagacaataaatggattacaa
ccttgcactatgctttccaggatgacaataacttatacctggttatggattattatgttggtggggatttgcttactctactc
agcaaatttgaagatagattgcctgaagatatggctagattttacttggctgagatggtgatagcaattgactcagttcatca
gctacattatgtacacagagacattaaacctgacaatatactgatggatatgaatggacatattcggttagcagattttggtt
cttgtctgaagctgatggaagatggaacggttcagtcctcagtggctgtaggaactccagattatatctctcctgaaatcctt
caagccatggaagatggaaaagggagatatggacctgaatgtgactggtggtctttgggggtctgtatgtatgaaatgcttta
cggagaaacaccattttatgcagaatcgctggtggagacatacggaaaaatcatgaaccacaaagagaggtttcagtttccag
cccaagtgactgatgtgtctgaaaatgctaaggatcttattcgaaggctcatttgtagcagagaacatcgacttggtcaaaat
ggaatagaagactttaagaaacacccatttttcagtggaattgattgggataatattcggaactgtgaagcaccttatattcc
agaagttagtagcccaacagatacatcgaattttgatgtagatgatgattgtttaaaaaattctgaaacgatgcccccaccaa
cacatactgcattttctggccaccatctgccatttgttggttttacatatactagtagctgtgtactttctgatcggagctgt
ttaagagttacggctggtcccacctcactggatcttgatgttaatgttcagaggactctagacaacaacttagcaactgaagc
ttatgaaagaagaattaagcgccttgagcaagaaaaacttgaactcagtagaaaacttcaagagtcaacacagactgtccaag
ctctgcagtattcaactgttgatggtccactaacagcaagcaaagatttagaaataaaaaacttaaaagaagaaattgaaaaa
ctaagaaaacaagtaacagaatcaagtcatttggaacagcaacttgaagaagctaatgctgtgaggcaagaactagatgatgc
ttttagacaaatcaaggcttatgaaaaacaaatcaaaacgttacaacaagaaagagaagatctaaataaggaactagtccagg
ctagtgagcgattaaaaaaccaatccaaagagctgaaagacgcacactgtcagaggaaactggccatgcaggaattcatggag
atcaatgagcggctaacagaattgcacacccaaaaacagaaacttgctcgccatgtccgagataaggaagaagaggtggacct
ggtgatgcaaaaagttgaaagcttaaggcaagaactgcgcagaacagaaagagccaaaaaagagctggaagttcatacagaag
ctctagctgctgaagcatctaaagacaggaagctacgtgaacagagtgagcactattctaagcaactggaaaatgaattggag
ggactgaagcaaaaacaaattagttactcaccaggagtatgcagcatagaacatcagcaagagataaccaaactaaagactga
tttggaaaagaaaagtatcttttatgaagaagaattatctaaaagagaaggaatacatgcaaatgaaataaaaaatcttaaga
aagaactgcatgattcagaaggtcagcaacttgctctcaacaaagaaattatgattttaaaagacaaattggaaaaaaccaga
agagaaagtcaaagtgaaagggaggaatttgaaagtgagttcaaacaacaatatgaacgagaaaaagtgttgttaactgaaga
aaataaaaagctgacgagtgaacttgataagcttactactttgtatgagaacttaagtatacacaaccagcagttagaagaag
aggttaaagatctagcagacaagaaagaatcagttgcacattgggaagcccaaatcacagaaataattcagtgggtcagcgat
gaaaaggatgcacgagggtatcttcaggccttagcttctaaaatgactgaagaattggaggcattaagaaattccagcttggg
tacacgagcaacagatatgccctggaaaatgcgtcgttttgcgaaactggatatgtcagctagactggagttgcagtcggctc
tggatgcagaaataagagccaaacaggccatccaagaagagttgaataaagttaaagcatctaatatcataacagaatgtaaa
ctaaaagattcagagaagaagaacttggaactactctcagaaatcgaacagctgataaaggacactgaagagcttagatctga
aaaggctagcaaaggcagacgtactgtagactccactccactttcagttcacacaccaaccttaaggaaaaaaggatgtcctg
gttcaactggctttccacctaagcgcaagactcaccagttttttgtaaaatcttttactactcctaccaagtgtcatcagtgt
acctccttgatggtgggtttaataagacagggctgttcatgtgaagtgtgtggattctcatgccatataacttgtgtaaacaa
agctccaaccacttgtccagttcctcctgaacagacaaaaggtcccctgggtatagatcctcagaaaggaataggaacagcat
atgaaggtcatgtcaggattcctaagccagctggagtgaagaaagggtggcagagagcactggctatagtgtgtgacttcaaa
ctctttctgtacgatattgctgaaggaaaagcatctcagcccagtgttgtcattagtcaagtgattgacatgagggatgaaga
attttctgtgagttcagtcttggcttctgatgttatccatgcaagtcggaaagatataccctgtatatttagggtcacagctt
cccagctctcagcatctaataacaaatgttcaatcctgatgctagcagacactgagaatgagaagaataagtgggtgggagtg
ctgagtgaattgcacaagattttgaagaaaaacaaattcagagaccgctcagtctatgttcccaaagaggcttatgacagcac
tctacccctcattaaaacaacccaggcagccgcaatcatagatcatgaaagaattgctttgggaaacgaagaagggttatttg
ttgtacatgtcaccaaagatgaaattattagagttggtgacaataagaagattcatcagattgaactcattccaaatgatcag
cttgttgctgtgatctcaggacgaaatcgtcatgtacgactttttcctatgtcagcattggatgggcgagagaccgattttta
caagctgtcagaaactaaagggtgtcaaaccgtaacttctggaaaggtgcgccatggagctctcacatgcctgtgtgtggcta
tgaaaaggcaggtcctctgttatgaactatttcagagcaagacccgtcacagaaaatttaaagaaattcaagtcccatataat
gtccagtggatggcaatcttcagtgaacaactctgtgtgggattccagtcaggatttctaagataccccttgaatggagaagg
aaatccatacagtatgctccattcaaatgaccatacactatcatttattgcacatcaaccaatggatgctatctgcgcagttg
agatctccagtaaagaatatctgctgtgttttaacagcattgggatatacactgactgccagggccgaagatctagacaacag
gaattgatgtggccagcaaatccttcctcttgttgttacaatgcaccatatctctcggtgtacagtgaaaatgcagttgatat
ctttgatgtgaactccatggaatggattcagactcttcctctcaaaaaggttcgacccttaaacaatgaaggatcattaaatc
ttttagggttggagaccattagattaatatatttcaaaaataagatggcagaaggggacgaactggtagtacctgaaacatca
gataatagtcggaaacaaatggttagaaacattaacaataagcggcgttattccttcagagtcccagaagaggaaaggatgca
gcagaggagggaaatgctacgagatccagaaatgagaaataaattaatttctaatccaactaattttaatcacatagcacaca
tgggtcctggagatggaatacagatcctgaaagatctgcccatgaaccctcggcctcaggaaagtcggacagtattcagtggc
tcagtcagtattccatctatcaccaaatcccgccctgagccaggccgctccatgagtgctagcagtggcttgtcagcaaggtc
atccgcacagaatggcagcgcattaaagagggaattctctggaggaagctacagtgccaagcggcagcccatgccctccccgt
cagagggctctttgtcctctggaggcatggaccaaggaagtgatgccccagcgagggactttgacggagaggactctgactct
ccgaggcattccacagcttccaacagttccaacctaagcagccccccaagcccagtttcaccccgaaaaaccaagagcctctc
cctggagagcactgaccgcgggagctgggacccgtga
SEQ ID NO: 2: (1732 aa)
MSGEVRLRQLEQFILDGPAQTNGQCFSVETLLDILICLYDECNNSPLRREKNILEYLEWAKPFTSKVKQMRLHRE
DFEILKVIGRGAFGEVAVVKLKNADKVFAMKILNKWEMLKRAETACFREERDVLVNGDNKWITTLHYAFQDDNNL
YLVMDYYVGGDLLTLLSKFEDRLPEDMARFYLAEMVIAIDSVHQLHYVHRDIKPDNILMDMNGHIRLADEGSCLK
LMEDGTVQSSVAVGTPDYISPEILQAMEDGKGRYGPECDWWSLGVCMYEMLYGETPFYAESLVETYGKIMNHKER
FQFPAQVTDVSENAKDLIRRLICSREHRLGQNGIEDFKKHPFFSGIDWDNIRNCEAPYIPEVSSPTDTSNFDVDD
DCLKNSETMPPPTHTAFSGHHLPFVGFTYTSSCVLSDRSCLRVTAGPTSLDLDVNVQRTLDNNLATEAYERRIKR
LEQEKLELSRKLQESTQTVQALQYSTVDGPLTASKDLEIKNLKEEIEKLRKQVTESSHLEQQLEEANAVRQELDD
AFRQIKAYEKQIKTLQQEREDLNKELVQASERLKNQSKELKDAHCQRKLAMQEFMEINERLTELHTQKQKLARHV
RDKEEEVDLVMQKVESLRQELRRTERAKKELEVHTEALAAEASKDRKLREQSEHYSKQLENELEGLKQKQISYSP
GVCSIEHQQEITKLKTDLEKKSIFYEEELSKREGIHANEIKNLKKELHDSEGQQLALNKEIMILKDKLEKTRRES
QSEREEFESEFKQQYEREKVLLTEENKKLTSELDKLTTLYENLSIHNQQLEEEVKDLADKKESVAHWEAQITEII
QWVSDEKDARGYLQALASKMTEELEALRNSSLGTRATDMPWKMRRFAKLDMSARLELQSALDAEIRAKQAIQEEL
NKVKASNIITECKLKDSEKKNLELLSEIEQLIKDTEELRSEKGIEHQDSQHSFLAFLNTPTDALDQFERSPSCTP
ASKGRRTVDSTPLSVHTPTLRKKGCPGSTGFPPKRKTHQFFVKSFTTPTKCHQCTSLMVGLIRQGCSCEVCGFSC
HITCVNKAPTTCPVPPEQTKGPLGIDPQKGIGTAYEGHVRIPKPAGVKKGWQRALAIVCDFKLFLYDIAEGKASQ
PSVVISQVIDMRDEEFSVSSVLASDVIHASRKDIPCIFRVTASQLSASNNKCSILMLADTENEKNKWVGVLSELH
KILKKNKFRDRSVYVPKEAYDSTLPLIKTTQAAAIIDHERIALGNEEGLFVVHVTKDEIIRVGDNKKIHQIELIP
NDQLVAVISGRNRHVRLFPMSALDGRETDFYKLSETKGCQTVTSGKVRHGALTCLCVAMKRQVLCYELFQSKTRH
RKFKEIQVPYNVQWMAIFSEQLCVGFQSGFLRYPLNGEGNPYSMLHSNDHTLSFIAHQPMDAICAVEISSKEYLL
CFNSIGIYTDCQGRRSRQQELMWPANPSSCCYNAPYLSVYSENAVDIFDVNSMEWIQTLPLKKVRPLNNEGSLNL
LGLETIRLIYFKNKMAEGDELVVPETSDNSRKQMVRNINNKRRYSFRVPEEERMQQRREMLRDPEMRNKLISNPT
NFNHIAHMGPGDGIQILKDLPMNPRPQESRTVFSGSVSIPSITKSRPEPGRSMSASSGLSARSSAQNGSALKREF
SGGSYSAKRQPMPSPSEGSLSSGGMDQGSDAPARDFDGEDSDSPRHSTASNSSNLSSPPSPASPRKTKSLSLEST
DRGSWDP
List below is amino acid sequence of NKA β1
(SEQ ID NO: 3, 303 aa)
MARGKAKEEGSWKKFIWNSEKKEFLGRTGGSWFKILLFYVIFYGCLAGIFIGTIQVMLLTISEFKPTYQDRVAPP
GLTQIPQIQKTEISFRPNDPKSYEAYVLNIVRFLEKYKDSAQRDDMIFEDCGDVPSEPKERGDFNHERGERKVCR
FKLEWLGNCSGLNDETYGYKEGKPCIIIKLNRVLGFKPKPPKNESLETYPVMKYNPNVLPVQCTGKRDEDKDKVG
NVEYFGLGNSPGFPLQYYPYYGKLLQPKYLQPLLAVQFTNLTMDTEIRIECKAYGENIGYSEKDRFQGRFDVKIE
VKS

### Improving Integrity or Function of Tight Junction

The Na⁺, K⁺-ATPase is well-known for its transport activity—moving Na⁺ out of the cell and importing K⁺. The results herein have identified new functions of this enzyme. Specifically, it was found that the small, non-catalytic β1 subunit promotes alveolar epithelial barrier integrity through a transport-independent mechanism that involves protein interaction and activation of protein kinase MRCKα (FIG. 8C). Inhibition of MRCKα using either siRNA or pharmacological inhibitors prevented the upregulation of occludin and the increase of TEER induced by β1 subunit overexpression; on the other hand, overexpression MRCKα alone was sufficient to enhance barrier functions. Consistent with an activation of MRCKα, overexpression of the β1 subunit increased the phosphorylation of myosin light chain kinase at Ser19 (32). Blebbistatin, a specific inhibitor of myosin-II ATPase activity abrogated the increase of TEER by β1 subunit overexpression. Together, these data demonstrate that the β1 subunit increases epithelial tight junction function by controlling MRCKα activation and myosin-actin activity.

During investigation to decipher the signaling pathway, this invention established a cellular model of alveolar epithelial barrier using ATI-like cells that enables efficient and dose-dependent induction of gene expression. Using this model, it was demonstrated that overexpression the β1 subunit led to improved barrier integrity, as demonstrated by the upregulation of tight junctions, increased electrical resistance, and decreased permeability to fluorescent tracers. To date, this is the first direct evidence supporting that the β1 subunit enhances epithelial cell barrier function in the lung. This study supplements existing data in mice and pigs (16, 18, 45), and provides a mechanistic basis to apply an ARDS gene therapy approach for human clinical use. The cellular model established here can be used to study other lung or pulmonary diseases characterized by barrier defects, such as asthma. Remarkably, electroporation was used to achieve high transfection efficiency, comparable to a previous study using nucleofection (46). Combined with tetracycline-inducible plasmids, this invention was able to achieve time- and dose-dependent gene expression even after cells were plated and have already formed a tight monolayer. This reduced the experimental variation generated during transfection of different plasmids, and allows measurement of barrier function in response to genetic perturbation without the use of viral vectors, which themselves have been shown to regulate the expression or the localization of tight junction proteins (47).

The data disclosed herein suggest that the β1 subunit upregulates tight junction specifically in ATI but not in ATII. This cell-type specificity warrants further investigation. One possibility is because of the presence of caveolae in ATI, but not in ATII, since the recycling of tight junctions requires caveolin-mediated endocytosis (42, 48). Another possibility is due to the disparity of MRCKα levels in these cell types. The data here suggest that the β1 improves barrier integrity via its interaction with MRCKα. Hence, a higher expression of MRCKα in ATI than ATII may explain their difference in changes of tight junctions upon β1 overexpression. Costaining of MRCKα and markers of ATI and ATII in lung sections is expected to test this hypothesis.

The data disclosed herein suggest that ion transport-activity is not required for the β1-mediated tight junction upregulation as demonstrated by ouabain treatment. Ouabain has diverse functions on the Na⁺, K⁺-ATPase depending on its concentration. At low concentrations (less than 20 nM), ouabain is insufficient to inhibit enough enzyme to alter intercellular Na⁺ and K⁺ levels, but affects a number of biological processes such as growth and gene expression through signaling (49). In contrast, at higher concentrations (greater than 100 nM), ouabain inhibits pump activity by inducing the internalization and lysosomal degradation of the Na⁺, K⁺-ATPase al subunit (50). The data here from ATI cells treated with both low and high concentrations of ouabain showed that transport activity alone is unable to explain the increased tight junction overexpression caused by β1 overexpression. The ion-independent regulation of tight junctions was further confirmed by using different forms of β subunit isoforms, which are all capable of forming functional complexes with the al subunit. The finding that only β1 upregulates occludin and zo-1 demonstrates that the β1 subunit has unique signaling functions separate from the other two isoforms. Surprisingly, overexpression of the β3 subunit, but not the β2 subunit decreased the β1 protein levels after 24 hours, resulting in lower occludin and zo-1 levels. This effect of overexpressing β3 mimics the effect of knocking down β1, further supports inventor's hypothesis that pump activity is not needed for the upregulation of tight junctions. It is worth mentioning that such a competing mechanism between β1 and β3 subunits, but not with the β2 subunit, has been reported in the literature (51). β1 knockout mice showed high β3 subunit expression (52) but overexpression of the β2 subunit in mice did not decrease β1 subunit levels (53). Experiments to compare the effect of the three subunits in treating LPS-induced lung injury will further substantiate the results disclosed herein. Given the data disclosed herein, it can be predicted that while gene transfer of the β1 subunit to mice with LPS-induced lung injury alleviates the severity of injury; similar transfer of the β2 subunit would not have any possible effects; similar transfer of the β3 subunit could even exacerbate the injury.

The results demonstrate that the β1 subunit-mediated tight junction upregulation is a process independent of the ion transport activity of the Na⁺, K⁺-ATPase. This is consistent with previous findings from inventor's laboratory (15, 16, 18) and others (12-14, 61) that only the β1 subunit, but not the α subunit nor the epithelial sodium channel, decreases lung permeability and treats mice with existing ARDS. Importantly, the finding here that the upregulation of tight junctions is exclusive to the β1 subunit, not the β2 or β3 isoform, further substantiates this conclusion since overexpression of the β2 or β3 subunits should also lead to increased ion transport activity but do not induce tight junctions.

Measurement of ion transport activity (either by ATP hydrolysis which is dependent on transport (62), or by uptake of ⁸⁶Rb⁺) will allow one to rule out the possibility that the β2 or β3 subunit was unable to form α1β2 or α1β3 complexes, or that these two complexes had lower ion transport-activity compared with α1β1. However, both possibilities are unlikely since the β2 and β3 subunit have been shown to form functional complex with the al subunit (63, 64), and indeed, the α1β2 complex has been reported to have even higher transport activity than the α1β1 complex (65). A chimera of β1 and β2 (replacing either the N-terminal cytoplasmic domain, or the C-terminal extracellular domain of β1 with the corresponding β2 sequence) would also further validate that the β1-mediated tight junction upregulation is independent on its transport activity but requires specific amino acid sequences.

The results presented here have confirmed some known protein interactions of the β1 subunit, including the Na⁺, K⁺-ATPase α1 subunit, the ER protein Wolframin (54), coatomer subunit β (55), and lethal giant larvae protein (56). Some proteins previously reported to interact with the β1 subunit (57-60) are not detected in the analysis disclosed herein, likely because these proteins—mainly expressed in the neural system—have low expression in the lung. More importantly, many new binding partners have been identified. To date, this is the first proteomic analysis of the β1 subunit interaction network. The interactome of many integral membrane proteins has remained unknown or is only poorly characterized due to their hydrophobicity, low expression, and lack of trypsin cleavage sites in their transmembrane segments (66, 67). This invention has greatly enriched the knowledge of protein interactions of the β1 subunit. The protein partners identified from this study can be confirmed by further experiments and provide important information regarding the activity and cellular functions of the Na⁺, K⁺-ATPase.

A previous study using siRNA-injection into mouse embryos proposed that the β1 subunit is required for proper distribution of tight junctions, likely via regulation of the actin cytoskeleton (30). The data presented here suggest that MRCKα appears to be involved in these processes. MRCKα is involved in cell migration, polarization and junction formation by regulating actin-myosin activity (30, 33, 68). MRCKα activation is increased by interacting with the β1 subunit. It could be that its association with the β1 subunit increases the plasma membrane localization of MRCKα, similar to that seen for β1 subunit with the sodium calcium exchanger 1 (69) or Megalencephalic leukoencephalopathy with subcortical cysts 1 (59). Another possibility is that the β1 association with MRCKα abolishes the auto-inhibition of MRCKα by binding to its two distal CC domains, which interact intramolecularly with the kinase domain and negatively regulate its activity (28). These two events may also happen concurrently.

One striking finding from results disclosed herein is that lungs from patients with ARDS tend to express lower amounts of MRCKα. No genetic susceptibility of ARDS has been linked to MRCKα so far. Yet, one of its downstream targets, myosin light chain kinase, is associated with ARDS susceptibility and outcomes (70). Additionally, a recent study suggested that MRCKα is involved in epithelial extrusion following apoptosis (71). Epithelial extrusion is a process by which dying or unwanted cells are removed from an epithelium while preserving the barrier function of the layer (72). To date, no study has explored the physiological and pathological roles of MRCKα in the lung. It will be quite interesting to investigate whether decreased MRCKα result in a defect of epithelial extrusion, thereby predisposing the lung to injuries that ultimately lead to ARDS.

The reason why lungs from ARDS patients express significantly lower amounts of MRCKα is unclear. One possibility is lower basal transcription of MRCKα due to genetic causes (such as reduced gene copy numbers or epigenetic modification). Another possibility is that risk factors for ARDS, such as inflammation, downregulate MRCKα levels. Regardless, MRCKα is a useful drug target for treating ARDS, or other human diseases characterized by barrier defects. Currently, only inhibitors of MRCKα have been identified (35, 73). Activation of MRCKα may be achieved by using a peptide that corresponds to the interacting domains on the Na⁺, K⁺-ATPase β1 subunit. Such a peptide modulator is also a promising drug to enhance epithelial barrier function and could ultimately lead to a simple pharmacological treatment of ARDS.

The data disclosed herein indicated a non-transport role of the Na⁺, K⁺-ATPase β1 subunit in the regulation of tight junctions. This invention has enhanced the understanding of the Na⁺, K⁺-ATPase and MRCKα and is valuable in advancing gene therapy to human clinical trials. Accordingly, this invention provides agents and methods for improving integrity or function of an epithelial or endothelial barrier. The methods in general comprise increasing a level of MRCKα in one or more cells in the epithelial or endothelial barrier. The method further comprises increasing a level of NKA β1. In certain aspect, the invention provides compositions and method for treating related diseases.

The present invention provides for both prophylactic and therapeutic methods of treating a subject at risk of (or susceptible to) a disorder or having a disorder associated with integrity or function of an epithelial or endothelial barrier. Another aspect of the invention pertains to methods of modulating MRCKα and/or NKA β1 expression or activity for therapeutic purposes.

Accordingly, in an exemplary embodiment, the modulatory method of the invention involves contacting a cell with an active agent or compound that modulates one or more of the activities of MRCKα and/or NKA β1 activity associated with the cell.

An active compound that modulates MRCKα and/or NKA β1 activity can be an agent as described herein, such as a nucleic acid or a protein, a naturally-occurring target molecule of an MRCKα protein (e.g., an MRCKα ligand or substrate), an MRCKα agonist or antagonist, a peptidomimetic of an MRCKα agonist or antagonist, or other small molecule. In one embodiment, the active compound stimulates one or more MRCKα activities. Examples of such stimulatory active compounds include active MRCKα protein and a nucleic acid molecule encoding MRCKα that has been introduced into the cell. In some embodiments, an active compound that modulates MRCKα and/or NKA β1 activity can be NKA β1 protein or polypeptide, or a nucleic acid molecule encoding NKA β1.

These modulatory methods can be performed in vitro (e.g., by culturing the cell with the active compound) or, alternatively, in vivo (e.g., by administering the active compound to a subject). As such, the present invention provides methods of treating an individual afflicted with a disease or disorder characterized by aberrant or insufficient expression or activity of an MRCKα protein or nucleic acid molecule such as a lung disorder. In one embodiment, the method involves administering an active compound, or combination of active compounds that modulates (e.g., upregulates) MRCKα and/or NKA β1 expression or activity. In another embodiment, the method involves administering a chimeric MRCKα and/or NKA β1 protein or nucleic acid molecule as therapy to compensate for reduced, aberrant, or unwanted MRCKα and/or NKA β1 expression or activity.

The present invention also provides for replacement of MRCKα and/or NKA β1, whether by gene transfer to express the normal allele or protein replacement with purified MRCKα and/or NKA β1 or recombinant MRCKα and/or NKA β1 or MRCKα and/or NKA β1 analogues, are beneficial for the treatment of, e.g., pulmonary disorders. The pathology of the lung disease includes acute lung injury, ARDS, and asthma. Other examples include idiopathic pulmonary fibrosis (IPF), desquamating interstitial pneumonitis (DIP), usual interstitial pneumonitis (UIP), non-specific interstitial pneumonitis (NSIP), and other forms of lung diseases, including inflammatory and hereditary lung diseases, such as cystic fibrosis, emphysema, pulmonary fibrosis, bronchiectasis, and recurrent infection.

The active agent, e.g., the MRCKα and/or NKA β1 gene or protein, may be administered by aerosol or inhalation of a pharmaceutically useful preparation containing surfactant-like phospholipids, including phosphatidylglycerol, phosphatidylcholine.

### Gene Therapy

As summarized above, one aspect of this invention includes a method of improving integrity or function of an epithelial or endothelial barrier, comprising increasing a level of MRCKα and/or NKA β1 in one or more cells in the epithelial barrier. Other aspects of the invention include methods of treating a disease or condition associated with compromised function of a epithelial or endothelial barrier comprising increasing a level of MRCKα and/or NKA β1 in one or more cells in the epithelial or endothelial barrier of a subject in need thereof.

In one embodiment, methods are provided for supplying MRCKα and/or NKA β1 function to cells of the lung and airway, such as smooth muscle, epithelial cells, and endothelial cells, by gene therapy. The MRCKα and/or NKA β1 genes, a modified MRCKα and/or NKA β1 gene, or a part of the gene may be introduced into the cell in a vector such that the gene remains extrachromosomal or may be integrated into the subjects chromosomal DNA for expression. These methods provide for administering to a subject in need of such treatment a therapeutically effective amount of an MRCKα and/or NKA β1 gene, or pharmaceutically acceptable composition thereof, for overexpressing the MRCKα and/or NKA β1 gene.

The MRCKα or NKA β1 gene or a part of the gene may or may not be integrated (covalently linked) to chromosomal DNA making up the genome of the subject's target cells. The genes may be introduced into the cell such that the gene remains extrachromosomal. In such a situation, the gene will be expressed by the cell from the extrachromosomal location. The cells may also be transformed where the exogenous DNA has become integrated into the chromosome so that it is inherited by daughter cells through chromosome replication. The gene may be introduced into an appropriate vector for extrachromosomal maintenance or for integration into the host. Vectors for introduction of genes both for recombination and for extrachromosomal maintenance are known in the art, and any suitable vector may be used. Methods for introducing DNA into cells such as electroporation, calcium phosphate co-precipitation and viral transduction are known in the art, and the choice of method is within the competence of those in the art.

The gene of the present invention as described herein is a polynucleotide or nucleic acid which may be in the form of RNA or in the form of DNA, which DNA includes cDNA, genomic DNA, and synthetic DNA. The DNA may be double-stranded or single-stranded, and if single stranded may be the coding strand or non-coding (anti-sense) strand. The coding sequence of MRCKα polynucleotide which encodes the mature polypeptide identified by SEQ ID NO: 2 may be identical or different from SEQ ID NO: 1. However, as a result of the redundancy or degeneracy of the genetic code, said coding sequence encodes the same mature polypeptide.

The polynucleotide or nucleic acid which encodes for the mature MRCKα or NKA β1 polypeptide may include: only the coding sequence for the mature polypeptide; the coding sequence for the mature polypeptide and additional coding sequence; the coding sequence for the mature polypeptide (and optionally additional coding sequence) and non-coding sequence, such as introns or non-coding sequence 5′ and/or 3′ of the coding sequence for the mature polypeptide.

The polynucleotide or nucleic acid compositions or molecules of this invention can include RNA, cDNA, genomic DNA, synthetic forms, and mixed polymers, both sense and antisense strands, and may be chemically or biochemically modified or may contain non-natural or derivatized nucleotide bases, as will be readily appreciated by those skilled in the art. Such modifications include, for example, labels, methylation, substitution of one or more of the naturally occurring nucleotides with an analog, internucleotide modifications such as uncharged linkages (e.g., methyl phosphonates, phosphotriesters, phosphoamidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.). Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule.

In vivo expression of MRCKα and/or NKA β1 transgenes can be carried out by injection of transgenes directly into a specific tissue, such as direct intratracheal, intramuscular or intraarterial injection of naked DNA or of DNA-cationic liposome complexes, or to ex vivo transfection of host cells, with subsequent reinfusion.

Multiple approaches for introducing functional new genetic material into cells, both in vitro and in vivo are known. These approaches include integration of the gene to be expressed into modified retroviruses; integration into non-virus vectors; or delivery of a transgene linked to a heterologous promoter-enhancer element via liposomes; coupled to ligand-specification-based transport systems or the use of naked DNA expression vectors. Direct injection of transgenes into tissue produces localized expression PCT/US90/01515 (Felgner et al.) is directed to methods for delivering a gene coding for a pharmaceutical or immunogenic polypeptide to the interior of a cell of a vertebrate in vivo. PCT/US90/05993 (Brigham) is directed to a method for obtaining expression of a transgene in mammalian lung cells following either iv or intratracheal injection of an expression construct. While most gene therapy strategies have relied on transgene insertion into retroviral or DNA virus vectors, lipid carriers, may be used to transfect the lung cells of the host.

The polynucleotides or nucleic acids described above may be produced by replication in a suitable host cell. Natural or synthetic polynucleotide fragments coding for a desired fragment can be incorporated into recombinant polynucleotide constructs, usually DNA constructs, capable of introduction into and replication in a prokaryotic or eukaryotic cell. Usually the polynucleotide constructs can be suitable for replication in a unicellular host, such as yeast or bacteria, but may also be intended for introduction to (with and without integration within the genome) cultured mammalian or plant or other eukaryotic cell lines.

The polynucleotides or nucleic acids may also be produced by chemical synthesis and may be performed on commercial, automated oligonucleotide synthesizers. A double-stranded fragment may be obtained from the single-stranded product of chemical synthesis either by synthesizing the complementary strand and annealing the strands together under appropriate conditions or by adding the complementary strand using DNA polymerase with an appropriate primer sequence.

Polynucleotide or nucleic acid constructs prepared for introduction into a prokaryotic or eukaryotic host may comprise a replication system recognized by the host, including the intended polynucleotide fragment encoding the desired polypeptide, and will preferably also include transcription and translational initiation regulatory sequences operably linked to the polypeptide encoding segment. Expression vectors may include, for example, an origin of replication or autonomously replicating sequence (ARS) and expression control sequences, a promoter, an enhancer and necessary processing information sites, such as ribosome-binding sites, RNA splice sites, polyadenylation sites, transcriptional terminator sequences, and mRNA stabilizing sequences. Secretion signals may also be included where appropriate, whether from a native MRCKα and/or NKA β1 protein or from other receptors or from secreted polypeptides of the same or related species, which allow the protein to cross and/or lodge in cell membranes, and thus attain its functional topology, or be secreted from the cell. Such vectors may be prepared by means of standard recombinant techniques well known in the art.

An appropriate promoter and other necessary vector sequences can be selected so as to be functional in the host, and may include, when appropriate, those naturally associated with MRCKα and/or NKA β1 genes. Many useful vectors are known in the art and may be obtained from such vendors as STRATAGENE, NEW ENGLAND BIOLABS, PROMEGA BIOTECH, and others. Promoters such as the trp, lac and phage promoters, tRNA promoters and glycolytic enzyme promoters may be used in prokaryotic hosts. Useful yeast promoters include promoter regions for metallothionein, 3-phosphoglycerate kinase or other glycolytic enzymes such as enolase or glyceraldehyde-3-phosphate dehydrogenase, enzymes responsible for maltose and galactose utilization, and others. Appropriate non-native mammalian promoters might include the early and late promoters from SV40 or promoters derived from murine Moloney leukemia virus, mouse tumor virus, avian sarcoma viruses, adenovirus II, bovine papilloma virus or polyoma. In addition, the construct may be joined to an amplifiable gene so that multiple copies of the gene may be made.

In one embodiment, the nucleic acid construct can include at least one promoter selected from the group consisting of RNA polymerase III, RNA polymerase II, CMV promoter and enhancer, SV40 promoter, an HBV promoter, an HCV promoter, an HSV promoter, an HPV promoter, an EBV promoter, an HTLV promoter, an HIV promoter, and cdc25C promoter, a cyclin a promoter, a cdc2 promoter, a bmyb promoter, a DHFR promoter and an E2F-1 promoter. In some embodiments, one can use an Ubiquitin C promoter for long-term expression.

According to one embodiment of the present invention, a method is provided of supplying MRCKα or NKA β1 function to cells of the lung and airway, such as smooth muscle and epithelial cells, by MRCKα or NKA β1 gene therapy. The MRCKα or NKA β1 gene, a modified MRCKα or NKA β1 gene, or a part of the gene may be introduced into the cell in a vector such that the gene remains extrachromosomal. In such a situation, the gene will be expressed by the cell from the extrachromosomal location.

In accordance with the present invention, there is provided a method of treating airway disease comprising the administration to a patient in need of such treatment a therapeutically effective amount of a nucleic acid encoding MRCKα and/or NKA β1, or pharmaceutically acceptable composition thereof. Aspects of the methods include administering to the subject a first nucleic acid alone or in a vector including a coding sequence for MRCKα and optionally a second nucleic alone or in a vector encoding an NKA β1 subunit polypeptide. In some cases, the first nucleic acid may include both coding sequences. Gene therapy methods that utilize the nucleic acid are also provided. Embodiments of the invention include compositions, e.g., nucleic acid alone or in vectors and kits, etc., that find use in the methods.

The methods may lead to increase the expression of MRCKα and/or NKA β1 gene when administered to subjects (e.g., mammals). Administration of the vectors to the subject may ameliorate one or more symptoms or markers of the disease or condition.

### Vectors

As disclosed herein, one aspect of the invention is a nucleic acid in a vector. Application of the subject vector to a subject, e.g., using any convenient method such as a gene therapy method, may result in expression of one or more coding sequences of interest in cells of the subject, to produce a biologically active product that may modulate a biological activity of the cell. In some cases, the vector is a nucleic acid vector comprising a coding sequence for MRCKα. In some cases, the nucleic acid vector comprises a coding sequence for one or more MRCKα and/or NKA β1.

In some instances, the vector comprises a coding sequence for MRCKα and/or NKA β1 suitable for use in gene therapy. Gene therapy vectors of interest include any kind of particle that comprises a polynucleotide fragment encoding the MRCKα and/or NKA β1 protein, operably linked to a regulatory element such as a promoter, which allows the expression of a functional MRCKα and/or NKA β1 protein demonstrating its activity in the targeted cells. In some cases, MRCKα is encoded by the nucleic acid sequence as set forth in SEQ ID NO: 1 or is an active fragment or functional equivalent of MRCKα. In some instances, the vector include a regulatory sequence which is a constitutive promoter such as the cytomegalovirus (CMV) promoter.

The MRCKα and/or NKA β1 sequence used in the gene therapy vector may be derived from the same species as the subject. Any convenient MRCKα and/or NKA β1 sequences, or fragments or functional equivalents thereof, may be utilized in the subject vectors, including sequences from any convenient animal, such as a primate, ungulate, cat, dog, or other domestic pet or domesticated mammal, rabbit, pig, horse, sheep, cow, or a human. For example, gene therapy in humans may be carried out using the human MRCKα and/or NKA β1 sequence.

Accordingly, nucleic acid molecules encoding MRCKα and/or NKA β1 and their analogs can be used for (i) improving integrity or function of an epithelial or endothelial barrier or (ii) treatment of disorders related to barrier dysfunction. Examples of the analogs can include MRCKα isoforms, mimetics, fragments, hybrid proteins, fusion proteins oligomers and multimers of the above, homologues of the above, regardless of the method of synthesis or manufacture thereof including but not limited to, recombinant vector expression whether produced from cDNA or genomic DNA, synthetic, transgenic, and gene activated methods.

### Polypeptides

In some embodiments, the present invention provides a method of introducing MRCKα and/or NKA β1 polypeptides into the cells. In one embodiment the MRCKα is human MRCKα. In one embodiment the human MRCKα has the amino acid sequence set out in SEQ ID NO: 2. The term “MRCKα” also denotes variants of the protein of SEQ ID NO: 2, in which one or more amino acid residues have been changed, deleted, or inserted, and which has comparable biological activity as the not modified protein, such as those reported herein. A number of splice variants of MRCKα are known in the art and result in slightly different translated proteins. Some of them may have difference in about 50 of their amino acid residues but the remainder are the same while some other variants produce slightly smaller proteins. These variants may have the same activity as SEQ ID NO: 1. Examples of such variants include NM_00136601 NM_001366019, NM_003607.3, NM_001366010.1, XM_017002581.2, and XM_011544307.3.

The specific activity of MRCKα can be determined by various assays known in the art or describer herein.

Amino acid sequence variants of MRCKα can be prepared by introducing appropriate modifications into the nucleotide sequence encoding the MRCKα, or by peptide synthesis. Such modifications include, for example, deletions from, and/or insertions into, and/or substitutions of residues within the amino acid sequences of the MRCKα. Any combination of deletion, insertion, and substitution can be made to arrive at the final construct, provided that the final construct possesses comparable biological activity to the human MRCKα.

As used herein, the term “conservative sequence modifications” refers to amino acid modifications that do not significantly affect or alter the activity of the MRCKα. Conservative amino acid substitutions are ones in which the amino acid residue is replaced with an amino acid residue having a similar side chain. Families of amino acid residues having similar side chains have been defined in the art.

Amino acid substitutions can be made, in some cases, by selecting substitutions that do not differ significantly in their effect on maintaining (a) the structure of the peptide backbone in the area of the substitution, (b) the charge or hydrophobicity of the molecule at the target sit; or (c) the bulk of the side chain. For example, naturally occurring residues can be divided into groups based on side-chain properties; (1) hydrophobic amino acids (norleucine, methionine, alanine, valine, leucine, and isoleucine); (2) neutral hydrophilic amino acids (cysteine, serine, threonine, asparagine, and glutamine); (3) acidic amino acids (aspartic acid and glutamic acid); (4) basic amino acids (histidine, lysine, and arginine); (5) amino acids that influence chain orientation (glycine and proline); and (6) aromatic amino acids (tryptophan, tyrosine, and phenylalanine). Substitutions made within these groups can be considered conservative substitutions. Examples of substitutions include, without limitation, substitution of valine for alanine, lysine for arginine, glutamine for asparagine, glutamic acid for aspartic acid, serine for cysteine, asparagine for glutamine, aspartic acid for glutamic acid, proline for glycine, arginine for histidine, leucine for isoleucine, isoleucine for leucine, arginine for lysine, leucine for methionine, leucine for phenylalanine, glycine for proline, threonine for serine, serine for threonine, tyrosine for tryptophan, phenylalanine for tyrosine, and/or leucine for valine. Exemplary substitutions are shown in the table below. Amino acid substitutions may be introduced into human MRCKα and the products screened for retention of the biological activity of human MRCKα.

| Original Residue | Exemplary Substitutions |
|---|---|
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; Norleucine |
| Leu (L) | Norleucine; Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Norleucine |

As used herein, “functional equivalent” of MRCKα refers to a nucleic acid molecule that encodes a polypeptide that has MRCKα activity or a polypeptide that has MRCKα activity. The functional equivalent may displays 50%, 60%, 70%, 80%, 90%, 95%, 98%, 99%, 100% or more activity compared to a parent MRCKα sequence (e.g., SEQ ID NO: 2). Functional equivalents may be artificial or naturally-occurring. For example, naturally-occurring variants of the sequence in a population fall within the scope of functional equivalent. MRCKα sequences derived from other species also fall within the scope of the term “functional equivalent”, e.g., a murine MRCKα sequence. In a particular embodiment, the functional equivalent is a nucleic acid with a nucleotide sequence having at least 75%, 80%, 85%, 90%, 95%, 96%, 97%, 98%, 99%, 99.5%, 99.9% identity to the parent sequence. In a further embodiment, the functional equivalent is a polypeptide with an amino acid sequence having at least 75%, 80%, 85%, 90%, 95%, 96%, 97%, 98%, 99%, 99.5%, 99.9% identity to a parent sequence. In the case of functional equivalents, sequence identity should be calculated along the entire length of the nucleic acid. Functional equivalents may contain one or more, e.g. 2, 3, 4, 5, 10, 15, 20, 30 or more, nucleotide insertions, deletions and/or substitutions when compared to a parent sequence.

The term “functional equivalent” also encompasses nucleic acid sequences that encode a MRCKα polypeptide with at least 75%, 80%, 85%, 90%, 95%, 96%, 97%, 98%, 99%, 99.5%, 99.9% sequence identity to the parent amino acid sequence, but that show little homology to the parent nucleic acid sequence because of the degeneracy of the genetic code.

As used herein, the term “active fragment” refers to a nucleic acid molecule that encodes a polypeptide that has MRCKα kinase activity or polypeptide that has MRCKα kinase activity, but which is a fragment of the nucleic acid as set forth in the parent polynucleotide sequence or the amino acid sequence as set forth in parent polypeptide sequence. An active fragment may be of any size provided that MRCKα kinase activity is retained or it has the catalytic domain A fragment will have at least 75%, 80%, 85%, 90%, 95%, 96%, 97%, 98%, 99%, 99.5%, 100% identity to the parent sequence along the length of the alignment between the shorter fragment and longer parent sequence.

Fusion proteins including these fragments can be comprised in the nucleic acid vectors needed to carry out the invention. For example, an additional 5, 10, 20, 30, 40, 50 or even 100 amino acid residues from the polypeptide sequence, or from a homologous sequence, may be included at either or both the C terminal and/or N terminus without prejudicing the ability of the polypeptide fragment to fold correctly and exhibit biological activity. Sequence identity may be calculated by any one of the various methods in the art, including for example BLAST (Altschul S F, Gish W, Miller W, Myers E W, Lipman D J (1990). “Basic local alignment search tool”. J Mol Biol 215 (3): 403-410) and PASTA (Lipman, D J; Pearson, W R (1985). “Rapid and sensitive protein similarity searches”. Science 227 (4693): 1435-41; http://fasta.bioch.virginia.edu/fasta www2/fasta list2.shtml) and variations on these alignment programs.

The polypeptides described in this application can be prepared by conventional methods known in the art.

Viral Vectors

Any convenient viruses may be utilized in delivering the vector of interest to the subject. Viruses of interest include, but are not limited to a retrovirus, an adenovirus, an adeno-associated virus (AAV), a herpes simplex virus and a lentivirus. Viral gene therapy vectors are well known in the art, see e.g., Heilbronn & Weger (2010) Handb Exp Pharmacal. 197:143-70. Vectors of interest include integrative and non-integrative vectors such as those based on retroviruses, adenoviruses (AdV), adeno-associated viruses (AAV), lentiviruses, pox viruses, alphaviruses, and herpes viruses.

In some cases, non-integrative viral vectors, such as AAV, may be utilized. In one aspect, non-integrative vectors do not cause any permanent genetic modification. The vectors may be targeted to adult tissues to avoid having the subjects under the effect of constitutive expression from early stages of development. In some instances, non-integrative vectors effectively incorporate a safety mechanism to avoid over-proliferation of MRCKα and/or NKA β1 expressing cells. The cells may lose the vector (and, as a consequence, the protein expression) if they start proliferating quickly.

Non-integrative vectors of interest include those based on adenoviruses (AdV) such as gutless adenoviruses, adeno-associated viruses (AAV), integrase deficient lentiviruses, pox viruses, alphaviruses, and herpes viruses. In certain embodiments, the non-integrative vector used in the invention is an adeno-associated virus-based non-integrative vector, similar to natural adeno-associated virus particles. Examples of adeno-associated virus-based non integrative vectors include vectors based on any AAV serotype, i.e., AAVI, AAV2, AAV3, AAV4, AAV5, AAV6, AAV7, AAV8, AAV9, AAVIO, AAVII and pseudotyped AAV. Vectors of interest include those capable of transducing a broad range of tissues at high efficiency, with poor immunogenicity and an excellent safety profile. In some cases, the vectors transduce post-mitotic cells and can sustain long-term gene expression (up to several years) both in small and large animal models of the related disorders.

### Pharmaceutical Compositions

In another aspect, the present invention provides pharmaceutical compositions containing a therapeutically effective amount of MRCKα and/or NKA β1, or nucleic acid sequences encoding MRCKα and/or NKA β1, and a pharmaceutically acceptable carrier. Preferably, the coding nucleic acid sequences are contained within an expression vector, such as plasmid DNA or virus. The pharmaceutical composition can be adapted for administration to the airways of the patient, e.g., nose, sinus, throat and lung, for example, as nose drops, as nasal drops, by nebulization as an inhalant, vaporization, or other methods known in the art. Administration can be continuous or at distinct intervals as can be determined by a person skilled in the art.

The pharmaceutical compositions can be formulated according to known methods for preparing pharmaceutically useful compositions. Furthermore, as used herein, the phrase “pharmaceutically acceptable carrier” means any of standard pharmaceutically acceptable carriers. The pharmaceutically acceptable carrier can include diluents, adjuvants, and vehicles, as well as implant carriers, and inert, non-toxic solid or liquid fillers, diluents, or encapsulating material that does not react with the active ingredients of the invention. Examples include, but are not limited to, phosphate buffered saline, physiological saline, water, and emulsions, such as oil/water emulsions. The carrier can be a solvent or dispersing medium containing, for example, ethanol, polyol (for example, glycerol, propylene glycol, liquid polyethylene glycol, and the like), suitable mixtures thereof, and vegetable oils. Formulations containing pharmaceutically acceptable carriers are described in a number of sources which are well known and readily available to those skilled in the art. For example, Remington's Pharmaceutical Sciences (Martin E W [1995] Easton Pennsylavania, Mack Publishing Company, 19.sup.th ed.) describes formulations that can be used in connection with the subject invention. Formulations suitable for parenteral administration include, for example, aqueous sterile injection solutions, which may contain antioxidants, buffers, bacteriostats, and solutes which render the formulation isotonic with the blood of the intended recipient; and aqueous and nonaqueous sterile suspensions which may include suspending agents and thickening agents. The formulations may be presented in unit-dose or multi-dose containers, for example sealed ampoules and vials, and may be stored in a freeze dried (lyophilized) condition requiring only the condition of the sterile liquid carrier, for example, water for injections, prior to use. Extemporaneous injection solutions and suspensions may be prepared from sterile powder, granules, tablets, etc. It should be understood that in addition to the ingredients particularly mentioned above, the formulations of the subject invention can include other agents conventional in the art having regard to the type of formulation in question.

The pharmaceutical compositions can be administered to a subject by any route that results in prevention or alleviation of symptoms associated with a disease or condition associated with compromised function of an epithelial or endothelial barrier. For example, the nucleic acid molecules can be administered parenterally, intravenously (I.V.), intramuscularly (I.M.), subcutaneously (S.C.), intradermally (I.D.), orally, intranasally, etc. Examples of intranasal administration can be by means of a spray, drops, powder or gel and also described in U.S. Pat. No. 6,489,306, US20180344816, US20060078558, US20080070858, US20180298057, and US20150313924, which are incorporated herein by reference in their entireties. One embodiment of the present invention is the administration of the composition as a nasal spray. However, other means of drug administrations are well within the scope of the present invention.

The MRCKα and/or NKA β polypeptide or encoding nucleic acid molecule can be administered and dosed in accordance with good medical practice, taking into account the clinical condition of the individual patient, the site and method of administration, scheduling of administration, patient age, sex, body weight, and other factors known to medical practitioners. The pharmaceutically “effective amount” for purposes herein is thus determined by such considerations as are known in the art. For example, an effect amount of the polypeptide or encoding nucleic acid molecule is that amount necessary to provide a therapeutically effective amount of MRCKα and/or NKA β1, when expressed in vivo. The amount of MRCKα and/or NKA β1 or encoding nucleic acid molecule must be effective to achieve improvement including but not limited to total prevention and to improved survival rate or more rapid recovery, or improvement or elimination of symptoms associated with the related disorders and other indicators as are selected as appropriate measures by those skilled in the art. In accordance with the present invention, a suitable single dose size is a dose that is capable of preventing or alleviating (reducing or eliminating) a symptom in a patient when administered one or more times over a suitable time period. One of skill in the art can readily determine appropriate single dose sizes for systemic administration based on the size of a mammal and the route of administration.

### Therapeutic Uses

Pharmaceutical compositions according to the invention can be generally administered systemically. Depending on the disorder to be treated, the pharmaceutical compositions described herein may be administered orally, parenterally (e.g., via intravenous, subcutaneous, intracutaneous, intramuscular, intraarticular, intraarterial, intrasynovial, intrasternal, intrathecal, intralesional or intracranial injection), topically, mucosally (e.g., rectally or vaginally), nasally, buccally, ophthalmically, via inhalation spray (e.g., delivered via nebulzation, propellant or a dry powder device) or via an implanted reservoir.

In certain embodiments, the disclosure provides methods of treating or preventing respiratory distress or respiratory disorders comprising administering an effective amount of a pharmaceutical composition comprising an active pharmaceutical agent disclosed herein to a subject in need thereof.

In certain embodiments, the subject is diagnosed with acute respiratory distress syndrome; alcoholic lung syndrome; sepsis-associated lung disorders; bacterial and viral pneumonia; ventilator induced lung injury; bronchopulmonary dysplasia (BPD); asthma; bronchial, allergic, intrinsic, extrinsic or dust asthma; chronic or inveterate asthma; late asthma or airways hyper-responsiveness; chronic obstructive pulmonary disease (COPD); bronchitis; emphysema; allergic rhinitis; or cystic fibrosis. Other examples of the respiratory disorder include, but are not limited to, such as a cold virus infection, bronchitis, pneumonia, tuberculosis, irritation of the lung tissue, hay fever and other respiratory allergies, asthma, bronchitis, simple and mucopurulent chronic bronchitis, unspecified chronic bronchitis (including chronic bronchitis NOS, chronic tracheitis and chronic tracheobronchitis), emphysema, other chronic obstructive pulmonary disease, asthma, status asthmaticus and bronchiectasis. Other respiratory disorders include allergic and non-allergic rhinitis as well as non-malignant proliferative and/or inflammatory disease of the airway passages and lungs. Non-malignant proliferative and/or inflammatory diseases of the airway passages or lungs means one or more of (1) alveolitis, such as extrinsic allergic alveolitis, and drug toxicity such as caused by, e.g., cytotoxic and/or alkylating agents; (2) vasculitis such as Wegener's granulomatosis, allergic granulomatosis, pulmonary hemangiomatosis and idiopathic pulmonary fibrosis, chronic eosinophilic pneumonia, eosinophilic granuloma and sarcoidoses.

In certain embodiments, the agent disclosed herein is administered in combination with other pharmaceutical agents such as antibiotics, anti-viral agents, anti-inflammatory agents, bronchodilators, or mucus-thinning medicines. Examples of such an agent include glucocorticoid receptor agonist (steroidal and non-steroidal) such as triamcinolone, triamcinolone acetonide, prednisone, mometasone furoate, loteprednol etabonate, fluticasone propionate, fluticasone furoate, fluocinolone acetonide, dexamethasone cipecilate, desisobutyryl ciclesonide, clobetasol propionate, ciclesonide, butixocort propionate, budesonide, beclomethasone dipropionate, alclometasone dipropionate; a p38 antagonist such as losmapimod; a phosphodiesterase (PDE) inhibitor such as a methylxanthanine, theophylline, and aminophylline; a selective PDE isoenzyme inhibitor, a PDE4 inhibitor and the isoform PDE4D, such as tetomilast, roflumilast, oglemilast, ibudilast, ronomilast; a modulator of chemokine receptor function such as vicriviroc, maraviroc, cenicriviroc, navarixin; a leukotriene biosynthesis inhibitor, 5-lipoxygenase (5-LO) inhibitor, and 5-lipoxygenase activating protein (FLAP) antagonist such as TA270 (4-hydroxy-1-methyl-3-octyloxy-7-sinapinoylamino-2(1H)-quinolinone) such as setileuton, licofelone, quiflapon, zileuton, zafirlukast, or montelukast; and a myeloperoxidase antagonist such as resveratrol and piceatannol.

Methods of administering the compositions and agents disclosed herein include, but are not limited to, pulmonary administration, e.g., by use of an inhaler or nebulizer, and formulation with an aerosolizing agent. See, e.g., US20180298057, U.S. Pat. Nos. 6,019,968; 5,985,200; 5,985,309; 5,934,272; 5,874,064; 5,855,913; 5,290,540; and 4,880,078; and PCT Publication Nos. WO 92/19244; WO 97/32572; WO 97/44013; WO 98/31346; and WO 99/66903. In a specific embodiment, it may be desirable to administer the pharmaceutical compositions locally to the area in need of treatment; this may be achieved by, for example, and not by way of limitation, local infusion, by injection, or by means of an implant, said implant being of a porous, non-porous, or gelatinous material, including membranes, such as sialastic membranes, or fibers. In certain embodiments, the aerosolizing agent or propellant is a hydrofluoroalkane, 1,1,1,2-tetrafluoroethane, 1,1,1,2,3,3,3-heptafluoropropane, propane, n-butane, isobutene, carbon dioxide, air, nitrogen, nitrous oxide, dimethyl ether, trans-1,3,3,3-tetrafluoroprop-1-ene, or combinations thereof. In certain embodiments, the disclosure contemplates oral administration.

For aerosol delivery in humans or other primates, the aerosol is generated by a medical nebulizer system that delivers the aerosol through a mouthpiece, facemask, etc. from which the mammalian host can draw the aerosol into the lungs. Various nebulizers are known in the art and can be used in the method of the present invention. The selection of a nebulizer system depends on whether alveolar or airway delivery (i.e., trachea, primary, secondary or tertiary bronchi, etc.), is desired. The particular nucleic acid composition is chosen that is not too irritating at the required dosage.

Nebulizers useful for airway delivery include those typically used in the treatment of asthma. Such nebulizers are also commercially available. The amount of compound used will be an amount sufficient to provide for adequate transfection of cells after entry of the DNA or complexes into the lung and airway and to provide for a therapeutic level of transcription and/or translation in transfected cells. A therapeutic level of transcription and/or translation is a sufficient amount to prevent, treat, or palliate a disease of the host mammal following administration of the nucleic acid composition to the host mammals lung, particularly the alveoli or bronchopulmonary and bronchiolopulmonary smooth muscle and epithelial cells of the trachea, bronchi, bronchia, bronchioli, and alveoli. Thus, an effective amount of the aerosolized nucleic acid preparation, is a dose sufficient to effect treatment, that is, to cause alleviation or reduction of symptoms, to inhibit the worsening of symptoms, to prevent the onset of symptoms, and the like. The dosages of the preset compositions that constitute an effective amount can be determined in view of this disclosure by one of ordinary skill in the art by running routine trials with appropriate controls. Comparison of the appropriate treatment groups to the controls will indicate whether a particular dosage is effective in preventing or reducing particular symptoms.

The total amount of nucleic acid delivered to a mammalian host will depend upon many factors, including the total amount aerosolized, the type of nebulizer, the particle size, breathing patterns of the mammalian host, severity of lung disease, concentration of the nucleic acid composition in the aerosolized solution, and length of inhalation therapy.

Despite the interacting factors, one of ordinary skill in the art will be able readily to design effective protocols, particularly if the particle size of the aerosol is optimized. Based on estimates of nebulizer efficiency, an effective dose delivered usually lies in the range of about 1 mg/treatment to about 500 mg/treatment, although more or less may be found to be effective depending on the subject and desired result. It is generally desirable to administer higher doses when treating more severe conditions. Generally, if the nucleic acid is not integrated into the host cell genome, the treatment can be repeated on an ad hoc basis depending upon the results achieved. If the treatment is repeated, the mammalian host is monitored to ensure that there is no adverse immune response to the treatment. The frequency of treatments depends upon a number of factors, such as the amount of nucleic acid composition administered per dose, as well as the health and history of the subject.

### Kits

The disclosure also provides kits, where the kits include one or more components employed in methods of the invention, e.g., vectors, as described herein. In some embodiments, the subject kit includes a vector (as described herein), and one or more components selected from a promoter, a virus, a cell, and a buffer. Any of the components described herein may be provided in the kits, e.g., cells, constructs (e.g., vectors) encoding for MRCKα and/or NKA β1, components suitable for use in expression systems (e.g., cells, cloning vectors, multiple cloning sites (MSC), bi-directional promoters, an internal ribosome entry site (IRES), etc.), etc. A variety of components suitable for use in making and using constructs, cloning vectors and expression systems may find use in the subject kits. Kits may also include tubes, buffers, etc., and instructions for use. The various reagent components of the kits may be present in separate containers, or some or all of them may be pre-combined into a reagent mixture in a single container, as desired.

In addition to the above components, the kits may further include instructions for practicing the subject methods. These instructions may be present in the kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another form of these instructions is a computer readable medium, e.g., diskette, compact disk (CD), hard drive etc., on which the information has been recorded. Yet another form of these instructions that may be present is a website address which may be used via the internet to access the information at a removed site.

Aspects of the invention include providing a virus particle that includes a nucleic acid vector, e.g., as described above. Any convenient virus particles may be utilized, and include viral vector particles described above.

Aspects of the invention include providing a cell that includes a nucleic acid vector. The cell that is provided with the vector of interest may vary depending on the specific application being performed. Target cells of interest include eukaryotic cells, e.g., animal cells, where specific types of animal cells include, but are not limited to: insect, worm or mammalian cells. Various mammalian cells may be used, including, by way of example, equine, bovine, ovine, canine, feline, murine, non-human primate and human cells. Among the various species, various types of cells may be used, such as epithelial, endothelial, pulmonary, hematopoietic, neural, glial, mesenchymal, cutaneous, mucosal, stromal, muscle (including smooth muscle cells), spleen, reticulo-endothelial, hepatic, kidney, gastrointestinal, fibroblast, and other cell types.

### Definitions

The term “gene therapy”, as used herein, refers to the transfer of genetic material (e.g., DNA or RNA) of interest into a host to treat or prevent a genetic or acquired disease or condition phenotype. The genetic material of interest encodes a product (e.g., a protein, polypeptide, peptide, or functional RNA) whose production in vivo is desired. For example, the genetic material of interest can encode a hormone, receptor, enzyme, polypeptide or peptide of therapeutic value. Two basic approaches to gene therapy have evolved: (1) ex vivo and (2) in vivo gene therapy. In ex vivo gene therapy, cells are removed from a patient and, while being cultured, are treated in vitro. Generally, a functional replacement gene is introduced into the cell via an appropriate gene delivery vehicle/method (transfection, transduction, homologous recombination, etc.) and an expression system as needed and then the modified cells are expanded in culture and returned to the host/patient. These genetically reimplanted cells have been shown to produce the transfected gene product in situ. In in vivo gene therapy, target cells are not removed from the subject, rather the gene to be transferred is introduced into the cells of the recipient organism in situ, that is within the recipient. Alternatively, if the host gene is defective, the gene is repaired in situ. These genetically altered cells have been shown to produce the transfected gene product in situ.

The terms “peptide,” “polypeptide,” and “protein” are used herein interchangeably to describe the arrangement of amino acid residues in a polymer. A peptide, polypeptide, or protein can be composed of the standard 20 naturally occurring amino acid, in addition to rare amino acids and synthetic amino acid analogs. They can be any chain of amino acids, regardless of length or post-translational modification (for example, glycosylation or phosphorylation).

A “recombinant” peptide, polypeptide, or protein refers to a peptide, polypeptide, or protein produced by recombinant DNA techniques; i.e., produced from cells transformed by an exogenous DNA construct encoding the desired peptide. A “synthetic” peptide, polypeptide, or protein refers to a peptide, polypeptide, or protein prepared by chemical synthesis. The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Within the scope of this invention are fusion proteins containing one or more of the afore-mentioned sequences and a heterologous sequence. A heterologous polypeptide, nucleic acid, or gene is one that originates from a foreign species, or, if from the same species, is substantially modified from its original form. Two fused domains or sequences are heterologous to each other if they are not adjacent to each other in a naturally occurring protein or nucleic acid.

A conservative modification or functional equivalent of a peptide, polypeptide, or protein disclosed in this invention refers to a polypeptide derivative of the peptide, polypeptide, or protein, e.g., a protein having one or more point mutations, insertions, deletions, truncations, a fusion protein, or a combination thereof. It retains substantially the activity to of the parent peptide, polypeptide, or protein (such as those disclosed in this invention). In general, a conservative modification or functional equivalent is at least 60% (e.g., any number between 60% and 100%, inclusive, e.g., 60%, 70%, 75%, 80%, 85%, 90%, 95%, 96%, 97%, 98%, and 99%) identical to a parent (e.g., SEQ ID NO: 2).

A nucleic acid or polynucleotide refers to a DNA molecule (e.g., a cDNA or genomic DNA), an RNA molecule (e.g., an mRNA), or a DNA or RNA analog. A DNA or RNA analog can be synthesized from nucleotide analogs. The nucleic acid molecule can be single-stranded or double-stranded, but preferably is double-stranded DNA. An “isolated nucleic acid” refers to a nucleic acid the structure of which is not identical to that of any naturally occurring nucleic acid or to that of any fragment of a naturally occurring genomic nucleic acid. The term therefore covers, for example, (a) a DNA which has the sequence of part of a naturally occurring genomic DNA molecule but is not flanked by both of the coding sequences that flank that part of the molecule in the genome of the organism in which it naturally occurs; (b) a nucleic acid incorporated into a vector or into the genomic DNA of a prokaryote or eukaryote in a manner such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein. The nucleic acid described above can be used to express the protein of this invention. For this purpose, one can operatively linked the nucleic acid to suitable regulatory sequences to generate an expression vector.

A vector refers to a nucleic acid molecule capable of transporting another nucleic acid to which it has been linked. The vector can be capable of autonomous replication or integrate into a host DNA. Examples of the vector include a plasmid, cosmid, or viral vector. The vector includes a nucleic acid in a form suitable for expression of the nucleic acid in a host cell. Preferably the vector includes one or more regulatory sequences operatively linked to the nucleic acid sequence to be expressed.

A “regulatory sequence” includes promoters, enhancers, and other expression control elements (e.g., polyadenylation signals). Regulatory sequences include those that direct constitutive expression of a nucleotide sequence, as well as tissue-specific regulatory and/or inducible sequences. The design of the expression vector can depend on such factors as the choice of the host cell to be transformed, the level of expression of protein or RNA desired, and the like. The expression vector can be introduced into host cells to produce a polypeptide of this invention. A promoter is defined as a DNA sequence that directs RNA polymerase to bind to DNA and initiate RNA synthesis. A strong promoter is one which causes mRNAs to be initiated at high frequency.

The term “operably-linked” or “operably-linked” is used herein to refer to an arrangement of flanking sequences wherein the flanking sequences so described are configured or assembled so as to perform their usual function. Thus, a flanking sequence operably-linked to a coding sequence may be capable of effecting the replication, transcription and/or translation of the coding sequence. For example, a coding sequence is operably-linked to a promoter when the promoter is capable of directing transcription of that coding sequence. A flanking sequence need not be contiguous with the coding sequence, so long as it functions correctly. Thus, for example, intervening untranslated yet transcribed sequences can be present between a promoter sequence and the coding sequence, and the promoter sequence can still be considered “operably-linked” to the coding sequence. Each nucleotide sequence coding for a polypeptide will typically have its own operably-linked promoter sequence.

“Expression cassette” as used herein means a nucleic acid sequence capable of directing expression of a particular nucleotide sequence in an appropriate host cell, which may include a promoter operably linked to the nucleotide sequence of interest that may be operably linked to termination signals. The coding region usually codes for a functional RNA of interest. The expression cassette including the nucleotide sequence of interest may be chimeric. The expression cassette may also be one that is naturally occurring but has been obtained in a recombinant form useful for heterologous expression. The expression of the nucleotide sequence in the expression cassette may be under the control of a constitutive promoter or of a regulatable promoter that initiates transcription only when the host cell is exposed to some particular stimulus. In the case of a multicellular organism, the promoter can also be specific to a particular tissue or organ or stage of development.

Such expression cassettes can include a transcriptional initiation region linked to a nucleotide sequence of interest. Such an expression cassette is provided with a plurality of restriction sites for insertion of the gene of interest to be under the transcriptional regulation of the regulatory regions. The expression cassette may additionally contain selectable marker genes.

“Coding sequence” refers to a DNA or RNA sequence that codes for a specific amino acid sequence. It may constitute an “uninterrupted coding sequence”, i.e., lacking an intron, such as in a cDNA, or it may include one or more introns bounded by appropriate splice junctions.

As used herein, the percent homology between two amino acid sequences is equivalent to the percent identity between the two sequences. The percent identity between the two sequences is a function of the number of identical positions shared by the sequences (i.e., % homology=#of identical positions/total #of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

As used herein, “treating” or “treatment” refers to administration of a compound or agent to a subject who has a disorder or is at risk of developing the disorder with the purpose to cure, alleviate, relieve, remedy, delay the onset of, prevent, or ameliorate the disorder, the symptom of the disorder, the disease state secondary to the disorder, or the predisposition toward the disorder. The terms “prevent,” “preventing,” “prevention,” “prophylactic treatment” and the like refer to reducing the probability of developing a disorder or condition in a subject, who does not have, but is at risk of or susceptible to developing a disorder or condition.

An effective amount refers to the amount of an active compound/agent that is required to confer a therapeutic effect on a treated subject. Effective doses will vary, as recognized by those skilled in the art, depending on the types of conditions treated, route of administration, excipient usage, and the possibility of co-usage with other therapeutic treatment.

The term “pharmaceutical composition” refers to the combination of an active agent with a carrier, inert or active, making the composition especially suitable for diagnostic or therapeutic use in vivo or ex vivo.

A “pharmaceutically acceptable carrier,” after administered to or upon a subject, does not cause undesirable physiological effects. The carrier in the pharmaceutical composition must be “acceptable” also in the sense that it is compatible with the active ingredient and can be capable of stabilizing it. One or more solubilizing agents can be utilized as pharmaceutical carriers for delivery of an active compound. Examples of a pharmaceutically acceptable carrier include, but are not limited to, biocompatible vehicles, adjuvants, additives, and diluents to achieve a composition usable as a dosage form. Examples of other carriers include colloidal silicon oxide, magnesium stearate, cellulose, and sodium lauryl sulfate.

A “subject” refers to a human and a non-human animal. Examples of a non-human animal include all vertebrates, e.g., mammals, such as non-human mammals, non-human primates (particularly higher primates), dog, rodent (e.g., mouse or rat), guinea pig, cat, and rabbit, and non-mammals, such as birds, amphibians, reptiles, etc. In one embodiment, the subject is a human. In another embodiment, the subject is an experimental, non-human animal or animal suitable as a disease model.

As used herein, “pulmonary disease” refers to disorders and conditions generally recognized by those skilled in the art as related to the constellation of pulmonary diseases characterized by emphysema, monocytic infiltrates, fibrosis, epithelial cell dysplasia, and atypical accumulations of intracellular lipids in type II epithelial cells and alveolar macrophages, regardless of the cause or etiology. These include, but are not limited “airway obstructive diseases” e.g., respiratory disorder, such as, airway obstruction, allergies, asthma, acute inflammatory lung disease, chronic inflammatory lung disease, chronic obstructive pulmonary dysplasia, emphysema, pulmonary emphysema, chronic obstructive emphysema, adult respiratory distress syndrome, bronchitis, chronic bronchitis, chronic asthmatic bronchitis, chronic obstructive bronchitis, and intestitial lung diseases.

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention.

The term “about” generally refers to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

### EXAMPLES

### Example 1 Material and Methods

This example descibes material and methods used in Examples 2-9 bellow.

Plasmids and siRNA

The plasmids used in this study were obtained from a range of sources. pCDNA3 and pCMV-EGFP plasmids were purchased from INVITROGEN (Carlsbad, Calif.). Mouse Na+, K+-ATPase β2 subunit and mouse Na+, K+-ATPase β3 subunit with Myc-DDK tag were obtained from ORIGENE (Rockville, Md.). The Tet-On 3G drug-inducible gene expression system was purchased from CLONTECH (Mountain View, Calif.). The human Na⁺, K⁺-ATPase β1 subunit-coding sequence was inserted into the pTRE3 G vector at the Sall and BamHI restriction enzyme sites. The human MRCKα plasmid was a gift from Dr. Paolo Armando Gagliardi from the University of Bern (31). Knockdown was carried out using the TRIFECTA DSIRNA Kit (IDT, Coralville, Iowa) according to manufacturer's instruction. 100 nM of siRNA was used for each one million cells.

### Antibodies and Inhibitors

The primary antibodies for western blot include anti-Na⁺, K⁺-ATPase β1 subunit (UPSTATE, #05-382), anti-occludin (INVITROGEN, #71-1500), anti-zo-1 (INVITROGEN, #61-7300), anti-zo-2 (INVITROGEN, #71-1400), anti-actin (SIGMA, #A2066), anti-GAPDH (MILIPORE, #CB1-001), anti-Na+, K+-ATPase β2 subunit (ABCAM, #ab185210), anti-DDK (ORIGENE, #TA50011-100), anti-MRCKα (SANTA CRUZ, #sc-374568), anti-MYPT1 (CELL SIGNALING, #2634S), anti-phospho-MYPT1 (Thr696, CELL SIGNALING, #5163S), anti-myosin light chain2 (CELL SIGNALING, #3672S), and anti-phospho-myosin light chain2 (Ser19, CELL SIGNALING, #3672S). The primary antibodies for immunofluorescence include anti-occludin-Alexa Fluor594 (INVITROGEN, #331594), anti-zo-1-Alexa Fluor594 (INVITROGEN, #339194), anti-MRCKα (FISHER, #PA1-10038). The inhibitor for MRCKα BDP5290 was purchased from AOBIOUS (Gloucester, M A). Myosin inhibitor blebbistatin was purchased from ABCAM.

### Primary Cell Isolation and Cell Culture

Primary rat alveolar epithelial type II cells were isolated using an IgG-panning approach as described by Dobbs (74). Briefly, lungs from Sprague Dawley rats (200-250 g) were surgically removed and perfused, lavaged, and treated with 1 mg/ml of elastase (WORTHINGTON BIOCHEMICAL, Lakewood, N.J.) to release the epithelial cells. Next, lung lobes were separated, cut, minced, filtered and spin down at 1500 rpm for 15 minutes. The cells were resuspended with DMEM without FBS and transferred into two IgG plates. After incubation at 37° C. for one hour, non-adhered cells (predominately ATII cells) were transferred to a new tube and centrifuged at 1500 rpm for 15 minutes. The cells were resuspended in DMEM containing 10% FBS and plated on fibronectin coated plates. To coat the plates with fibronectin, 20 μg/ml of fibronectin from bovine plasma (F1141, SIGMA-ALDRICH, St. Louis, Mo.) was added to 100 mm culture plates (using 3 ml) or the upper chamber of the transwell plates (using 400 μl). Plates were left at 37° C. for 3 hours. Residual solution was removed, and plates were dried in a tissue culture hood for at least 30 minutes before cells were added. 16HBE14o-human bronchial epithelial cells were cultured in Dulbecco's modified Eagle's medium as previously reported (18).

### Transfection

Transfection was carried out by electroporation using the GENE PULSER MXCELL electroporation system (BIORAD, Hercules, Calif.). The condition for ATI cells was one square wave pulse at 300 V, 1000Ω, and 20 milliseconds.

### Western Blot

Cells were lysed with reporter lysis buffer (lx, PROMEGA), supplemented with protease inhibitor (COMPLETE, Mini, EDTA-free tablets; ROCHE, Basel, Switzerland) and phosphatase inhibitor (PHOSSTOP Phosphatase Inhibitor Cocktail; ROCHE, Basel, Switzerland). Proteins were separated on 10% SDS-PAGE, transferred to PVDF membrane, and probed with primary antibodies at room temperature for 2 hours or at 4° C. overnight. After incubation with secondary antibody and development, bands were detected on film (BIOMAX MR film; CARESTREAM HEALTH, Rochester, N.Y.) or using the CHEMIDOC IMAGING SYSTEM (BIO-RAD, Hercules, Calif.) and quantified using the IMAGE STUDIO™ LITE software (LI-COR, Lincoln, Nebr.) or the IMAGE LAB SOFTWARE (BIO-RAD, Hercules, Calif.).

qPCR

Total RNA was isolated using the RNeasy Mini Kit (QIAGEN, Hilden, Germany) After determining RNA concentration by spectrophotometry, 100 to 1000 ng of total RNA was used for cDNA synthesis. Reverse transcription was conducted using the REVERSE TRANSCRIPTION SYSTEM (PROMEGA, Madison, Wis.). Ten microliters of the reaction was diluted to 100 μl, from which one microliter was taken for quantitative real-time PCR using ITAQ™ UNIVERSAL SYBR® GREEN SUPERMIX (BIORAD, Hercules, Calif.). The specificity of primers was confirmed by melting curve analysis and gel electrophoresis. qPCR was performed on a CFX CONNECT REAL TIME PCR DETECTION SYSTEM (BIORAD, Hercules, Calif.). Samples were assayed in triplicate. Relative RNA level was quantified using the ΔΔCt method (75) and normalized to the endogenous control GAPDH unless specified otherwise.

### Immunofluorescence

Cells were washed three times before fixation with 4% paraformaldehyde in PBS for 15 minutes at room temperature. Fixed cells were washed with PBS and permeabilized with 0.2% Triton X-100 in PBS for 10 minutes. After washing with PBS, transwell inserts were blocked with blocking reagent (DAKO PROTEIN BLOCK SERUM FREE, AGILENT) for one hour and incubated with primary antibody at 4° C. overnight. Nuclei were stained with 2.5 μg/ml DAPI for 5 minutes, then washed twice with PBS. The transwell membrane was then carefully cut out using a clean razor blade and mounted on a glass slide WITH PROLONG ANTIFADE mounting media (FISHER, Waltham, Mass.). Slides were examined under a Leica DMI6000 microscope and photos were captured using the open source software MANAGER OR VOLOCITY SOFTWARE (VELOCITY INC.). Tissue sections of human lungs from patients with ARDS were provided by the department of Pathology at the University of Rochester using an Institutional Review Board approved protocol. All samples were taken at autopsy. In total, 16 sections from 6 ARDS patients and 7 sections from three control patients without ARDS were obtained. The H&E staining of each corresponding section shows varying degree of lung injury and edema content. For immunofluorescence staining, tissue sections were deparaffinized and rehydrated. Then, an antigen retrieval step was performed to expose epitopes for subsequent antibody binding and immunofluorescence.

### TEER

Prior to the assay, cells cultured on 12-well transwell plates (12 mm transwell with 0.4 μm pore polyester membrane insert; CORNING, Corning, N.Y.) were moved to the tissue culture hood for 15 minutes to allow the medium equilibrate to room temperature. TEER was measured using an epithelial voltmeter (EVOM2; WORLD PRECISION INSTRUMENTS, Sarasota, Fla.). Three readings were recorded and averaged for each well. To calculate TEER, the resistance of the fibronectin-coated insert without cells (blank resistance) was subtracted from the measured resistance, then multiplied by 1.12 cm² to account for the surface area of the insert.

### Permeability

Permeability to fluorescent tracers was measured using a modified protocol previous described (76). After TEER measurement, the upper and lower transwell chamber were washed twice with P buffer (10 mM HEPES at pH 7.4, 1 mM sodium pyruvate, 10 mM glucose, 3 mM CaCl₂, and 145 mM NaCl). Five hundred microliters of freshly prepared solution containing 100 μg/mL of 40 kD FITC-dextran and 100 μg/mL of 31(D Texas Red-dextran were added to the apical compartment. One thousand microliters of P buffer was added to the bottom chamber. After 2 hours incubation at 37° C., 100 μl of the basal medium was collected and the fluorescence of the transported dextran was measured with a SPECTRAMAX M5 multi-mode microplate reader (MOLECULAR DEVICES, San Jose, Calif.). The excitation wavelength and emission wavelength are 492 nm and 520 nm for FITC and 596 nm and 615 nm for Texas-red, respectively. The quantity of tracer was calculated by comparison with a standard curve. A permeability coefficient was determined using the following equation (77): Pc (cm/min)=V/(A×Co)×(C/T) where V is volume in the lower compartment (1 ml), A is the surface area of the membrane (1.12 cm² for the 12-well transwell used here), Co is the dextran concentration in the upper compartment at time 0 (0.1 mg/ml), and C is the dextran concentration in the lower compartment at time T of sampling (2 hours).

### Immunoprecipitation and Mass Spectrometry

Cells from one 100-mm plate were lysed with 1 ml of IP lysis buffer (1% NP-40, 50 mM Tris HCl pH 8.0) and homogenized 10 times with a 25-gauge syringe. Immunoprecipitation was performed using the μMACS Protein G Kit according to the manufacturer's instructions (MILTENYI BIOTEC, Bergisch-Gladbach, Germany) The precleared samples were incubated with anti-MRCKα antibody (PA1-10038, 1:50 dilution; FISHER, Waltham, Mass.), anti-01 antibody (UPSTATE, 05-382, 1:250 dilution) or IgG as control at 4° C. overnight. The elute was analyzed by a SDS-PAGE Gradient Gels (4-20%). Each lane was cut into 10 pieces of approximately the same size. The gel bands were then destained, reduced and digested with tripsin overnight. The digested peptide mixtures were then subjected to LC-MS/MS analysis using the Orbitrap system.

Label Free Quantification of Proteins Interacting with the β1 Subunit

Thermo raw data were transformed into mgf format. The resulting peak lists were searched using PROTEIN PROSPECTOR (v5.22.0) with the following settings: Trypsin as protease with a maximum of one missed cleavage sites, 10 ppm mass tolerance for MS, 0.5 Da (ion trap) and 0.05 Da (ORBITRAP), respectively for MS/MS, carbamidomethylation (C) as fixed, oxidation (M) as well as phosphorylation (S/T/Y) as variable modifications. Results from PROTEIN PROSPECTOR were retrieved and cleaned up using in-house python script. Protein quantitation using NSAF measurement was described previously (23). Data normalization, annotation and statistical analysis were performed using Perseus (78). Student's t test was used for statistical analysis of NSAF (79).

### Example 2 β1 Subunit Overexpression Increases Expression of Alveolar Tight Junctions

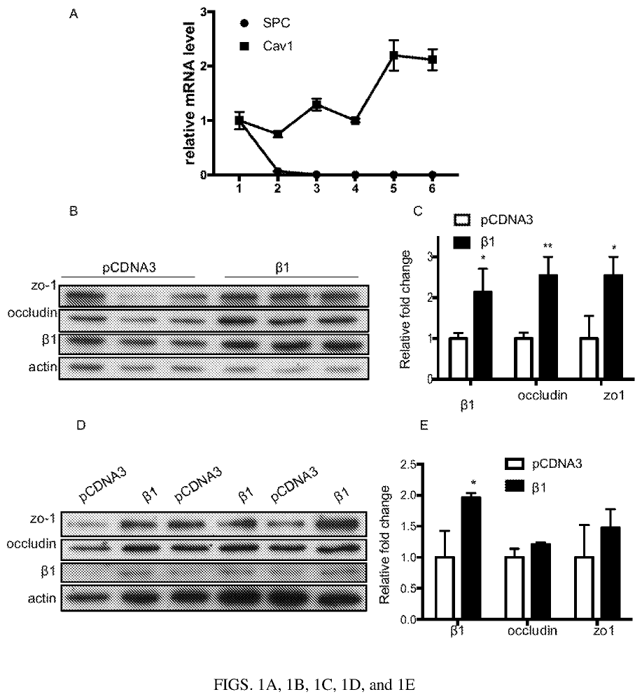

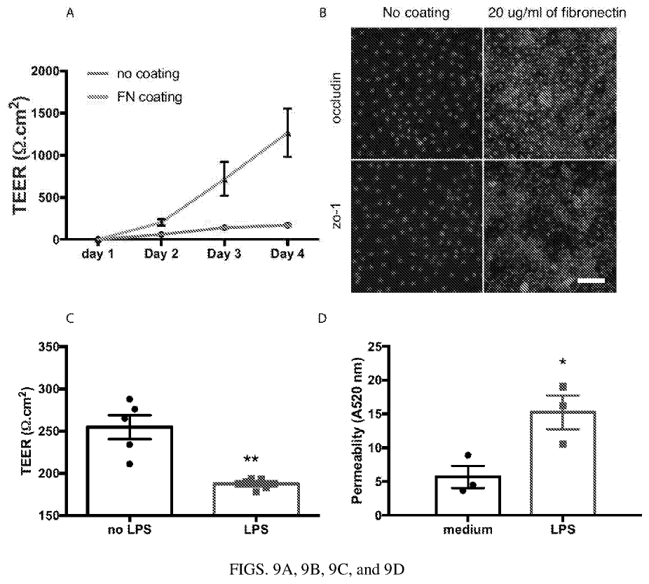

The majority of cell junctions in alveolar epithelial barrier are between adjacent ATI cells, which cover 95% of the its surface (5). Unfortunately, existing cell lines do not fully recapitulating the genetic and phenotypic characteristics of ATI in vivo and isolating ATI cells directly poses technical challenges (19). To overcome this, rat primary ATII cells were used since they are capable of differentiating into ATI cells when isolated and cultured in vitro (20). To track the phenotypic changes during the process, qPCR analysis was carried out for genes that are specific for ATII (SPC) or ATI (T1a) (FIG. 1A). SPC mRNA levels dropped 15-fold from 24 hours to 48 hours after isolation, suggesting a loss of ATII phenotype. In contrast, T1α level increased continuously until day five after isolation, indicating a shift to ATI phenotype. When cultured in transwell plates coated with 20 μg/ml fibronectin, these cells exhibited TEER, a measurement of electrical resistance across a cellular monolayer, comparable or higher than measured in 16HBE14o- or Calu-3 cell (FIG. 9A) (21), with near-continuous staining of occludin and zo-1 localized to the cell membrane (FIG. 9B). Meanwhile, when treated with 1 μg/ml of lipopolysaccharide, these cells showed decreased TEER and increased permeability to 4 kD Dextran (FIG. 9C and FIG. 9D), suggesting injuries to the epithelial barrier. These data indicate that this primary rat ATI culture system can serve as a relevant model to study alveolar epithelial barrier.

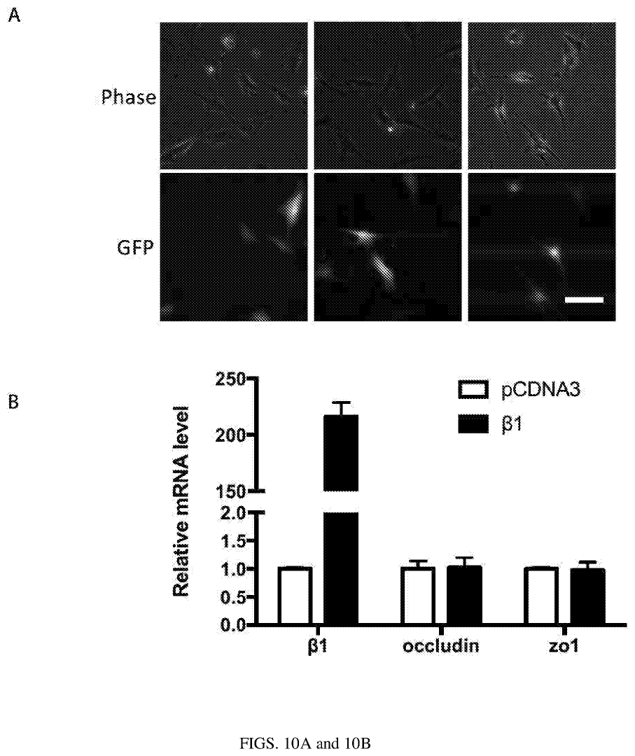

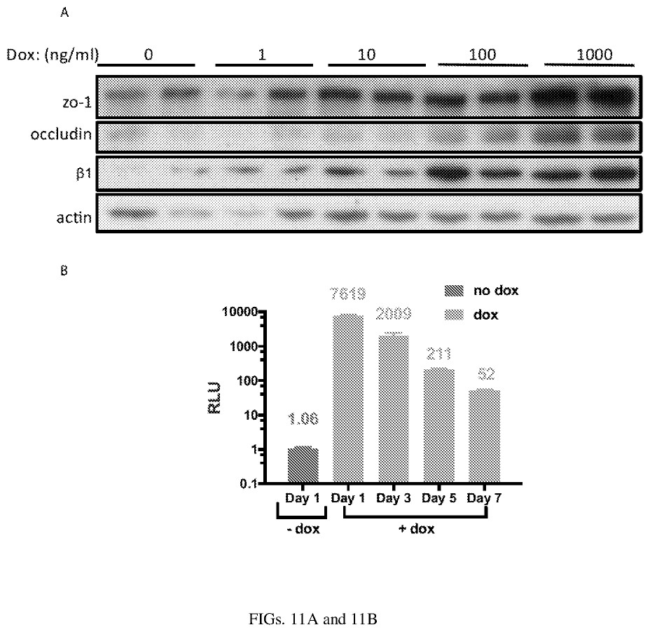

Next, the β1 subunit was overexpressed in ATI cells in order to examine its function on the epithelial barrier. Lipid-based approach did not result in detectable transfection in these cells, however, electroporation using a square wave of 300 V and 20 milliseconds resulted in about 50% transfection efficiency with minimal cell death, as determined by transfection of an EGFP-expressing plasmid (FIG. 10A). Using the same approach, ATI cells were transfected with a plasmid encoding the rat β1 subunit, and measured expression of tight junctions 24 hours later. At mRNA levels, overexpression of the β1 subunit had no effect on occludin or zo-1, despite increased level of the β1 subunit (FIG. 10B). At protein level, however, a 3.9-fold increase of occludin, 2.5-fold increase of zo-1, and 8.2-fold increase of zo-2 were observed (FIG. 1B and FIG. 1C). Surprisingly, when the β1 subunit was transfected into ATII cells, none of these tight junction proteins showed increased expression (FIG. 1D and FIG. 1E). Similar result was observed for A549 cells, a human ATII cell line (data not showing). These data demonstrate that the β1 subunit increases expression of tight junctions at protein levels, and such effect is specific for ATI cells in the alveoli.

### Example 3 Overexpression of β1 Subunit Increases Alveolar Type I Barrier Integrity

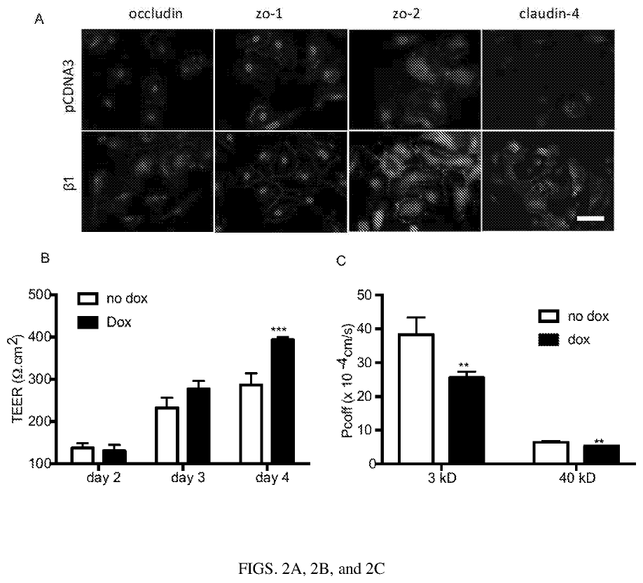

Given that the β1 subunit increased protein expressions of tight junctions in ATI cells, their localization in these cells was analyzed. Immunofluorescence staining confirmed increased level of occludin, zo-1, zo-2 and claudin-4 on the cell membrane (FIG. 2A). Next, assays were carried out to investigate the functional effect of the β1 subunit on the alveolar epithelial barrier. Since electroporation requires trypsinizing the cells, which would disrupt the cell monolayer and impede TEER and permeability assays, a doxycycline-inducible system was created to control the β1 subunit expression by cloning the rat β1 subunit into a Tet-on plasmid. This would allow one to turn on the gene expression in ATI cells at a later point after electroporation by just adding doxycline to the cell culture media. To test whether this system can be used to control the expression of the β1 subunit, 16HBE14o-cells, a human bronchial epithelial cell line, were cotransfected with pCMV-tet regulator plasmids and pTet3G-human β1 subunit expressing plasmids by electroporation, followed immediately by addition of an increasing concentrations of doxycycline (0, 1, 10, 100, 1000 ng/ml). Immunoblot analysis showed that doxycycline caused a dose-dependent upregulation of β1 subunit at 24 hours post transfection with maximum induction at 1000 ng/ml (FIG. 11A). Consequently, the expressions of occludin and zo-1 were also increased, confirming previous findings using the pCMV-rat β1 plasmid. Besides, using a luciferase reporter plasmid revealed that a transient transfection led to gene upregulation up to 7 days after transfection (FIG. 11B).

Using this system, assays were carried out to examine the role of the β1 subunit on tight junction function in ATI cells. Before doxycycline was added to the media, TEER had no significant difference among the cell monolayers (FIG. 10B). However, after adding 1 μg/ml of doxycycline, TEER was significantly higher at the doxycline-treated group (day 3 and day 4), suggesting an increase in barrier integrity. In line with the TEER measurement, the permeability to 31(D dextran and 40 kD dextran decreased by 33.2% and 18.5%, respectively, following doxycycline treatment (FIG. 2C). Taken together, these data have demonstrated that overexpression of the β1 subunit leads to improved alveolar epithelial barrier function.

### Example 4 β1 Subunit Mediated Tight Junction Upregulation is Ion-Transport Independent

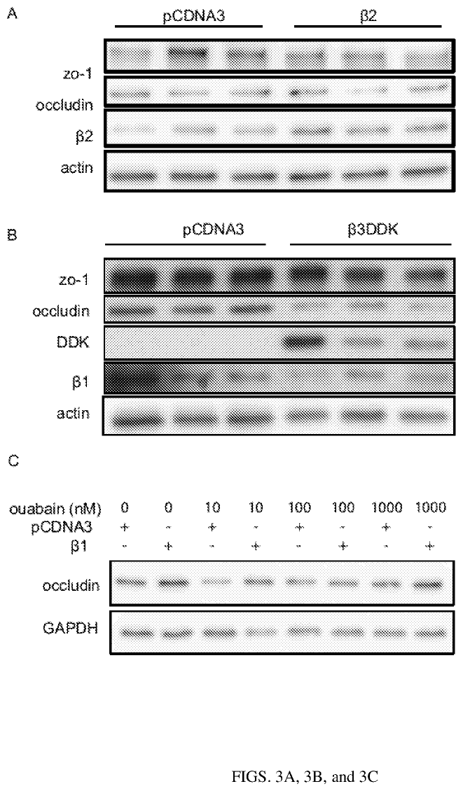

The β subunit of the Na⁺, K⁺-ATPase facilitates the maturation and membrane trafficking of the α subunit, thereby increasing ion transport activity (22). If this activity was required for the barrier-enhancing effect of the β1 subunit, overexpression of the β2 or β3 isoform could have the same effect as that the β1. To test this, ATI cells were transfected with plasmids encoding the mouse β2 subunit or the mouse β3 subunit three days after isolation when they displayed an ATI phenotype. Then the levels of tight junction proteins was evaluated by western blot after 24 hours. In contrast to the β1 subunit, overexpression of the β2 isoform did not increase the expression of occludin or zo-1 (FIG. 3A); overexpression of the β3 subunit even decreased their levels (FIG. 3B).

Surprisingly, it was found that overexpression of the β3 subunit decreased the total levels of the β1 subunit, possibly suggesting a competitive binding of these two β isoforms to the α subunit. To further confirm that pump activity is not mediating the barrier-enhancing effect, cells were treated with ouabain, a cardiac glycoside that inhibits ATP-dependent sodium-potassium exchange, following transfection with the β1 subunit. Immunoblot analysis showed that ouabain did not block the upregulation of occludin at any of the concentrations tested (FIG. 3C). Taken together, these results indicate that the β1 subunit promotes tight junction barrier function through a transport-independent mechanism.

### Example 5 MRCKα is a β1-Interacting Protein that Regulates Epithelial Barrier Integrity

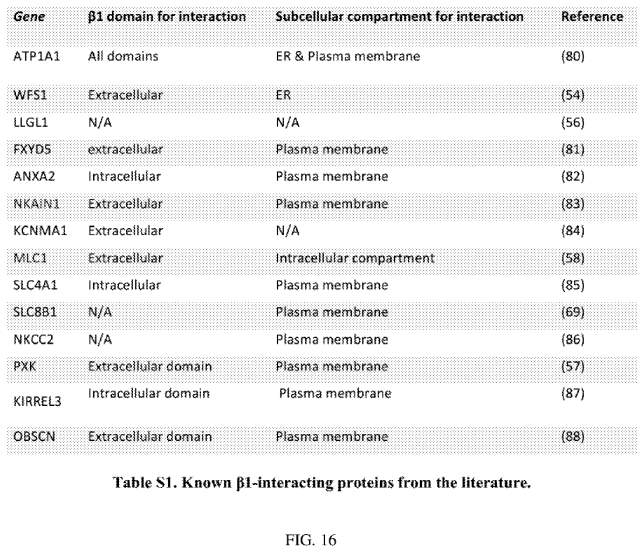

Since the above findings have suggested that the β1 subunit mediated epithelial barrier tightening is independent of its ion-transport activity, it was hypothesized that β1-interacting proteins may play a role. However, only a few of these proteins were reported in the literature and most of them are expressed only in the neural system (Table S1/FIG. 16). To systematically identify proteins that are interacting with the β1 subunit in the lung epithelial cells, tandem mass spectrometry was used.

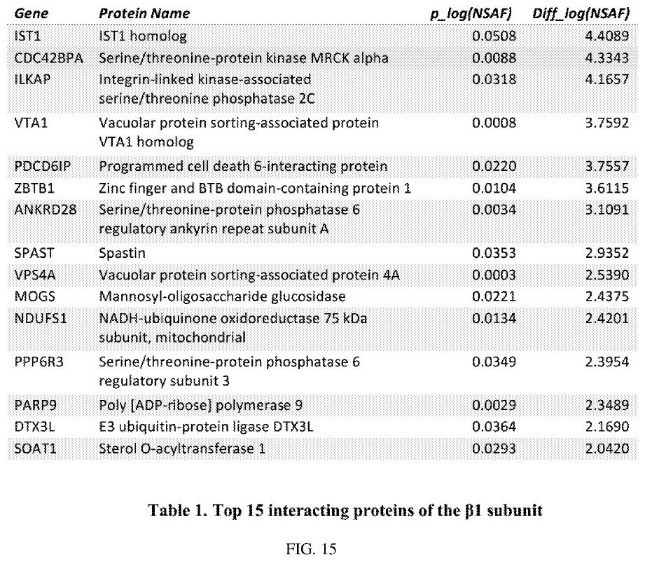

Cell lysates from 16HBE14o-cells were immunoprecipitated using protein G magnetic beads and an antibody against the β1 subunit or an antibody against GFP as negative control. The resulting protein complexes were then gel-purified and subjected to trypsin digestion. After database searching for the spectrums, 2936 unique proteins were identified from three independent experiments (supplementary file). Their relative abundances were then quantified using normalized spectrum abundance factor (NSAF), a label-free quantification method based on counting the number of unique peptides assigned to each protein (23). A total of 138 proteins passed the criterial for potential interactions (p<0.05, student's t test). Gene Ontology (GO) enrichment analysis (24, 25) of these proteins revealed significant enrichment for biological process including endosomal sorting complex required for transport (ESCRT) disassembly and multivesicular body organization, two processes involved in the endosomal sorting of ubiquitylated membrane proteins. Table 1 (FIG. 15) lists top 15 of the interacting proteins. Some interactors may play important roles for the regulation of β1 itself. For example, MOGS (Mannosyl-oligosaccharide glucosidase) may be involved in its N-glycosylation; DTX3L (E3 ubiquitin-protein ligase DTX3L) in its ubiquitination.

Next, proteins whose GO contains the term “cell junction” were further examined. These interactors of the β1 subunit may mediate its function in promoting alveolar epithelial barrier integrity. Indeed, two of the top 15 interactors have a GO term for “cell junction”: PDCD6IP (Programmed cell death 6-interacting protein or Alix) and CDCl42BPA (Myotonic dystrophy kinase-related CDCl42-binding kinase alpha or MRCKα). Alix is involved in the assembly of the actomyosin-tight junction polarity complex and the maintenance of epithelial barrier integrity. However, loss of Alix affects the organization, rather than the protein abundance of tight junctions (26). Thus, MRCKα was further studied.

MRCKα is a serine/threonine-protein kinase and a downstream effector of Cdc42 in cytoskeletal reorganization (27). At its native state, MRCKα forms a homodimer that blocks its kinase activity (28). Once activated, it phosphorylates substrates including myosin light chain kinase 2 and LIM kinase, thereby modulating actin-myosin contraction (29). The dissociation of the autoinhibitory dimerization is a prerequisite for MRCKα activation, which can be induced by a number of factors, such as Rap1 (30) and PDK1 (31). By regulating the cytoskeleton, activated MRCKα is involved in many cellular processes, such as cell migration (31, 32), cell polarity (33), and endothelial junction formation (30, 34). It was hypothesized that the β1 subunit may increase alveolar epithelial barrier integrity through MRCKα.

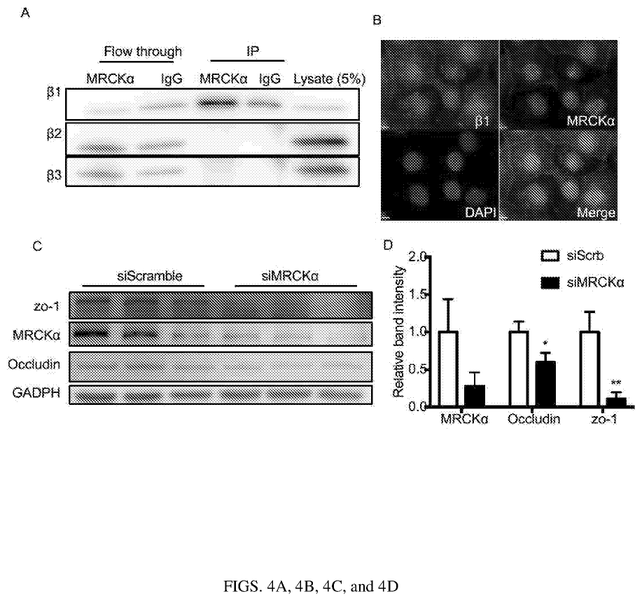

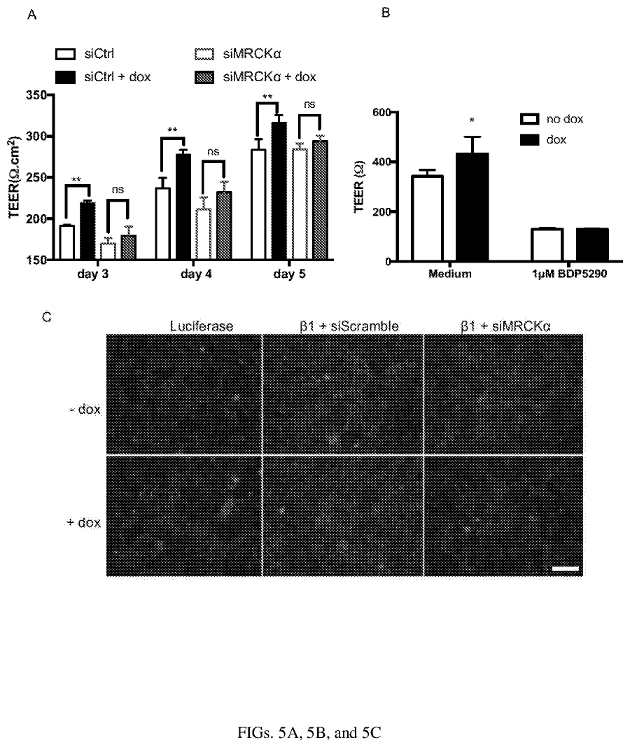

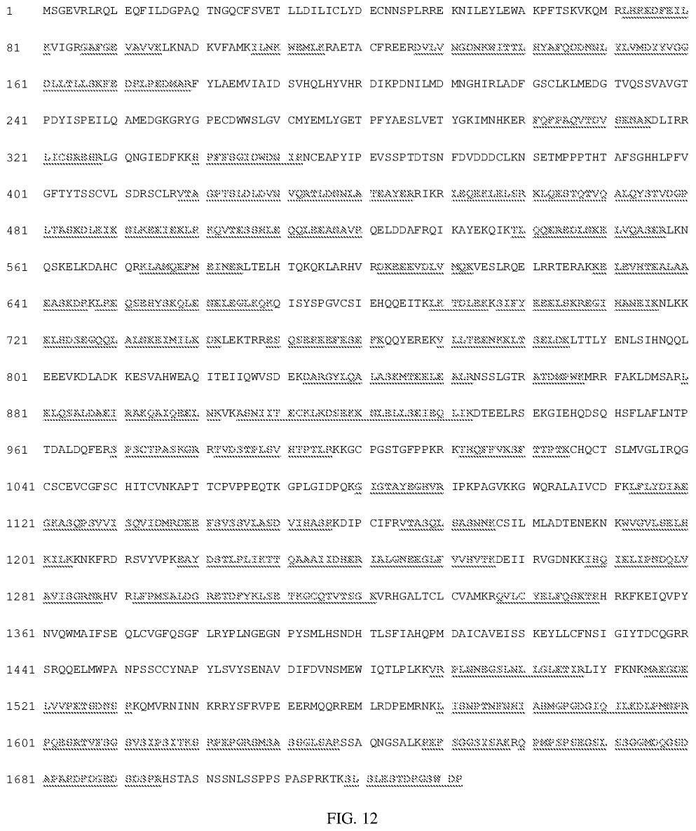

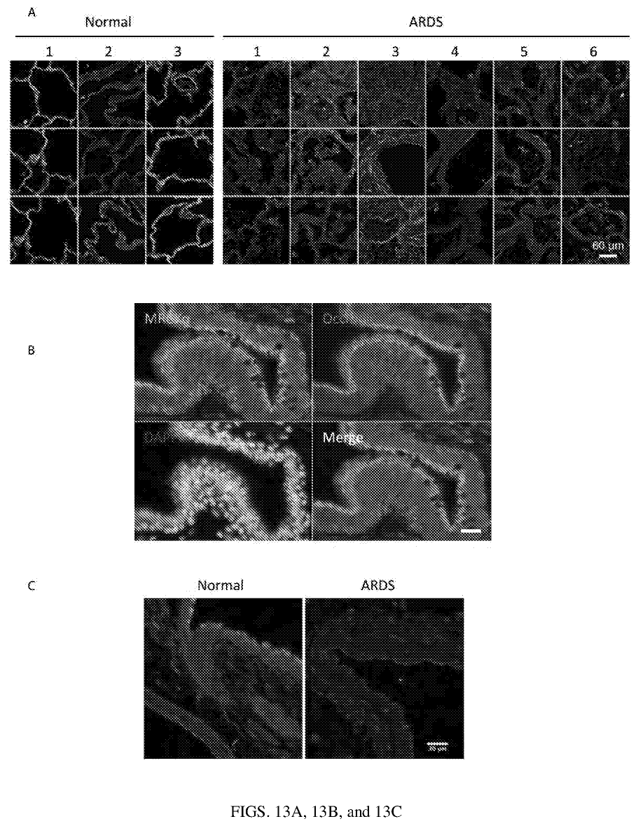

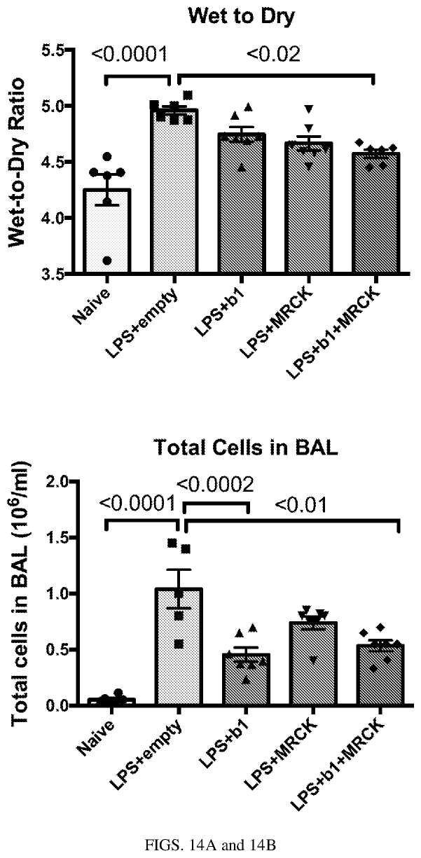

Although MRCKα has over 40% sequence coverage in the mass spectrometry analysis (FIG. 12), assays were carried out to confirm its interaction with the β1 subunit by coimmunoprecipitation experiment. Among the three β isoforms, only the β1 subunit was detected in the MRCKα pulldown complex, suggesting the specificity of this interaction (FIG. 4A). Further immunofluorescence staining in ATI cells showed that β1 colocalizes with MRCKα on the cell membrane (FIG. 4B). To decipher the role of MRCKα in epithelial barrier function, its expression was knocked down in ATI cells, and protein levels of tight junctions evaluated. Cells transfected with small interference RNA (siRNA) against MRCKα showed significantly lower levels of both occludin and zo-1 (FIG. 4C and FIG. 4D), suggesting that MRCKα may stabilize the expression of tight junction proteins.

Since MRCKα loss-of-function impaired tight junctions, it was hypothesized that the β1 subunit enhances alveolar barrier function though its interaction with MRCKα. To test this hypothesis, MRCKα was first knockdown using siRNA, and subsequently β1 overexpression was induced using doxycycline. TEER were significantly higher in ATI monolayer at 24, 48, and 72 hours after adding doxycycline, but was abolished when cells were transfected with siRNA against MRCKα (FIG. 5A). To further confirm this, cells were treated with 2 μM BDP5290, a potent inhibitor of MRCKα (35), and barrier integrity was evaluated with TEER. Consistent with siRNA knockdown, the baseline TEER was decreased upon MRCKα inhibition. More importantly, inhibitor treatment prevented β1 subunit-induced increase of barrier integrity (FIG. 5B) Immunofluorescence staining also confirmed that the β1 subunit promoted the membrane organization of zo-1, a finding that was not observed when MRCKα is knocked down (FIG. 5C). Collectively, these data indicated a critical role of the β1 subunit in improving alveolar barrier function through activation of MRCKα.

### Example 6 Overexpression of MRCKα Alone can Improve Alveolar Barrier Function

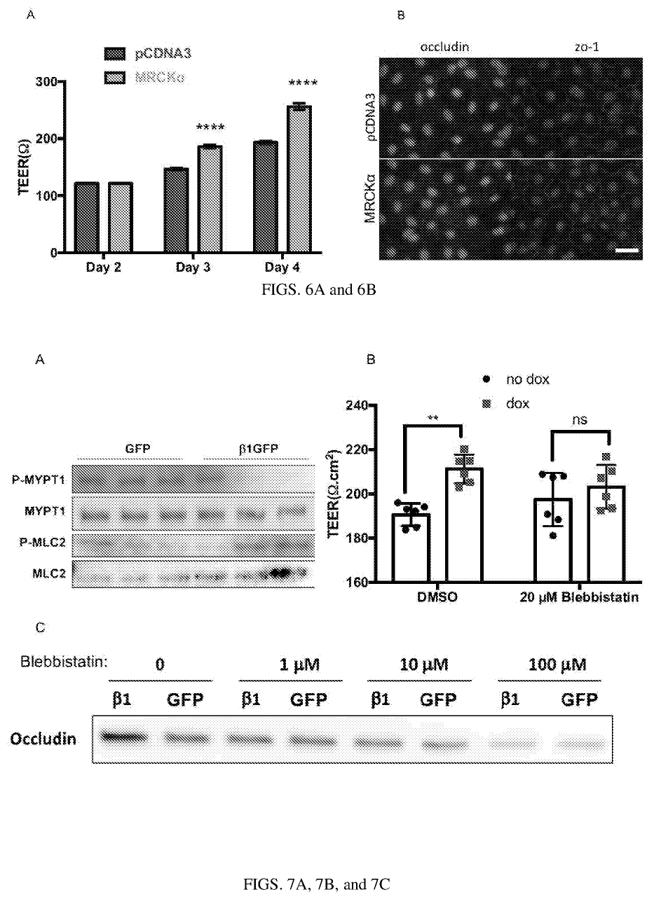

Since the above data suggest that MRCKα was a downstream mediator of the β1-induced potentiation of the ATI cell epithelial barrier, it was speculated that increasing the activity of MRCKα alone was sufficient to improve alveolar barrier function. To test this hypothesis, ATII cells were transfected with MRCKα plasmids, and barrier integrity was measured using TEER. At 24 hours after transfection, no significant differences in TEER was detected; however, at both 48 and 72 hours after transfection, significantly higher values were observed in cells transfected with MRCKα compared with empty plasmid control (FIG. 6A). In consistent with this result, occludin and zo-1 displayed higher intensities and increased localization to cell-cell border upon MRCKα transfection (FIG. 6B). These results demonstrate that overexpression of MRCKα alone is sufficient to promote alveolar epithelial barrier integrity.

### Example 7 Activation of Non-Muscle Myosin II Mediates β1 Subunit Stabilization of Tight Junctions

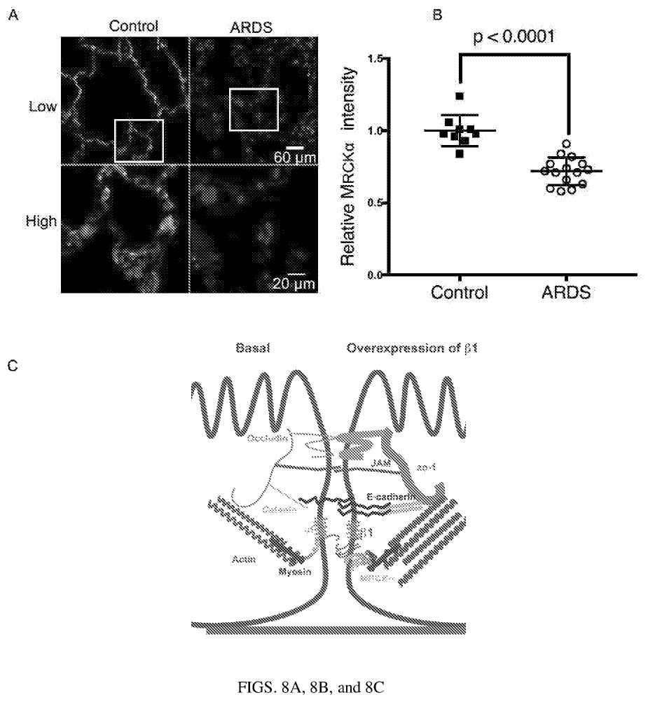

The results so far support the hypothesis that the β1 subunit interacting protein MRCKα is both necessary and required to promote alveolar tight junctions. To further substantiate this conclusion, assays were carried out to examine the activation of MRCKα downstream pathways, which include myosin light chain kinase 2 (directly or indirectly by inhibition of myosin phosphatase MYPT1), LIM kinases, and myoesin (29-31, 33, 36-38). The phosphorylation of these substrates were assessed using western blot analysis. It was found that β1 subunit overexpression induced the phosphorylation of myosin light chain 2 (MLC2) at Ser19 by 2-fold (FIG. 7A). Therefore, the data further confirm that the β1 subunit interacts and activates MRCKα.

The activation of actin-myosin regulates the assembly of tight junction complexes (39) and their stead state level through endocytic degradation (40-42). Activation of MLC2 promotes junctional recruitment, formation of circumferential actin bundles and barrier maturation (30, 33, 34, 43, 44). Therefore, assays were carried out to investigate whether β1-mediated activation of MLC2 is responsible for the increased barrier integrity. Pretreatment of 20 μM blebbistatin, a specific inhibitor of myosin II, prevented the increase in TEER induced by overexpression of the β1 subunit (FIG. 7B). Consistent with this result, western blot analysis showed that treatment of ATI cells with blebbistatin significantly suppress the upregulation of occludin (FIG. 7C). Taken together, these results suggest that the activation of myosin II is required for the β1-mediated tight junction stabilization and alveolar epithelial barrier potentiation.

### Example 8 Human ARDS Patients Show Decreased Expression of MRCKα

Given that MRCKα regulates alveolar barrier integrity, whether its expression alters in ARDS was investigated. Immunofluorescence staining demonstrated that lungs from ARDS patients express much lower level of MRCKα compared with lungs from control donors (FIG. 13A and FIG. 8A), with average 30% less relative fluorescent intensities (FIG. 8B). In addition to the alveoli, small airways also expressed high levels of MRCKα, especially in the cilia where occludin was expressed, and in the basal cells (FIG. 13B). Importantly, staining intensities in these tissues were also decreased in ARDS patients (FIG. 13C). Taken together, these data indicate that lower levels of MRCKα in the lung is associated with ARDS pathology.

### Example 9 Role of MRCKα In Vivo

In this example, assays were carried out to examine the roles of MRCKα in vivo. Briefly, the lungs of mice were injured with LPS, which mimics pneumonia infection or bacterial sepsis that causes acute lung injury/acute respiratory distress syndrome (ALI/ARDS). One day later, when the lungs were filled with neutrophils and pulmonary edema fluid, plasmids encoding various proteins as indicated in FIGS. 14A and 14B were delivered to the lung by electroporation. The plasmids include a control plasmid (“empty”, which expressed no gene product, hence a good negative control for added DNA), and those encoding the β1 subunit of the Na,K-ATPase (“b1”), MRCKα (“MRCK”), or a combination of β1 subunit and MRCKα (“b1+MRCK”). Two days after that, the lungs were harvested and examined for endpoints of injury.

The endpoints were wet-to-dry ratios of the lung histology, and the total number of infiltrating immune cells in the bronchioalveolar lavage fluid (BALF). A wet-to-dry ratio is a measure of pulmonary edema—the higher the ratio, the more water or edema in the lung, and thus the greater lung injury. The BALF is an indicator as to how injured the lung is and a high number of cells indicates a severe injury. The data (FIGS. 14A and 14B) show that gene transfer of MRCKα to lungs alone had a bit of an effect, but when delivered with the β1 subunit, the effect was more pronounced and highly statistically significant. This indicated that MRCK can be used alone to treat ALI/ARDS and in combination with the β1 subunit to give the best treatment. These results indicated that MRCKα works in vivo.

### Example 10 MRCKα Improves Alveolar-Capillary Epithelial-Endothelial Barrier Function

In this example, additional assays were carried out to examine the roles of MRCKα to improves alveolar-capillary epithelial-endothelial barrier function in vivo.

### Methods and Materials

Plasmids

The plasmid pcDNA3 was from PROMEGA (Madison, Wis.). pCMV-MRCKα expresses human MRCKα from the CMV promoter and pCMV-Na⁺,K⁺-ATPase β1 expresses a GFP-tagged rat Na⁺,K⁺-ATPase β1 subunit as described previously (Gagliardi, P. A., et al., J Cell Biol 206: 415-34, and Machado-Aranda, D., et al., Am J Respir Crit Care Med 171: 204-11). Plasmids were purified using QIAGEN GIGA-PREP KITS (QIAGEN, Chatsworth, Calif.) and suspended in 10 mM Tris-HCl (pH 8.0), 1 mM ethylenediaminetetraacetic acid, and 140 mM NaCl.

In-Vivo Gene Transfer and Induction of Acute Lung Injury

Male C57BL/6 mice (9-11 weeks) were anesthetized with isoflurane and 100 μg of each individual plasmid were delivered in 50 μl of 10 mM Tris-HCl (pH 8.0), 1 mM EDTA, and 140 mM NaCl, to mouse lungs by aspiration (when both β1 and MRCKα were delivered, 100 μg of each were administered prior to electroporation) as previously described in Lin, X., et al., Gene Ther 23: 489-99. Eight, 10 msec square wave pulses at a field strength of 200 V/cm were immediately applied using cutaneous electrophysiology electrodes (MEDTRONIC, Redmond, Wash.) placed on the mouse chest with an ECM830 electroporator (BTX, HARVARD APPARATUS, Holliston, Mass.). All LPS-challenged mice received 5 mg/kg of LPS (Escherichia coli 055:B5, 15,000,000 endotoxin units/mg protein; SIGMA-ALDRICH, St. Louis, Mo.) in 50 μl of phosphate-buffered saline (PBS) by aspiration, one day before gene transfer (n=5-11 mice/group). All experimental procedures were performed accordance with institutional guidelines for the care and use of laboratory animals in an American Association for the Accreditation of Laboratory Animal Care-approved facility.

Measurement of Alveolar Fluid Clearance (AFC) in Live Mice

The method used in this study was performed in live mice as previously described in Lin, X., et al., Gene Ther 23: 489-99 and Mutlu, G. M., et al., Circ Res 94: 1091-100. Briefly, mice maintained at a body temperature of 37° C. were anesthetized with diazepam (5 mg/kg, i.p.) and pentobarbital (50 mg/kg, i.p. given 10 minutes after diazepam). The trachea was cannulated with a 5-mm, 20-gauge angiocath (BECTON-DICKENSON, Sandy, Utah), and the catheter was connected to a small animal ventilator (HARVARD APPARATUS, Holliston, Mass.) before paralysis with pancuronium bromide (0.04 mg, i.p.). Mice were ventilated with 100% oxygen and a tidal volume of 10 nal/kg at a frequency of 160 breaths per minute. Three hundred ml of an isosmolar (324 mOsm), 0.9% NaCl solution containing 5% acid-free Evans Blue-labeled bovine serum albumin (0.15 mg/ml, SIGMA, St. Louis, Mo.) was instilled into the endotracheal catheter over 10 seconds followed by 200 μl of air to position the fluid in the alveolar space. Mice were kept supine, inclined to 30°, and ventilated for 30 minutes, after which the chest was opened to produce bilateral pneumothoraces to facilitate aspiration of fluid from the tracheal catheter. Protein concentration in the aspirate was assessed using a Bradford assay (BIO-RAD LABORATORIES, Hercules, Calif.) and AFC was calculated using following equation: AFC=1−(C₀/C₃₀), where C₀, is the protein concentration of the instillate before instillation, and C₃₀ is the protein concentration of the sample obtained at the end of 30 minutes of mechanical ventilation. Clearance is expressed as a percentage of total instilled volume cleared/30 minutes. Procaterol (a specific β₂AR agonist, 10⁻⁸ M) was administered in the instillate as positive control.

Measurement of Wet-to-Dry Ratios

The effect of LPS-induced acute lung injury on total lung water content was determined at 72 hours after instillation of LPS. Mice were exsanguinated via laceration of left renal artery and vein, and then lungs were excised and surface liquid was blotted away. Wet lung weight was assessed and a stable dry weight was obtained after lungs were placed in a hybridization oven at 70° C. for 72 h.

Bronchoalveolar Lavage (BAL) Analysis

BAL was performed as described previously in Lin, X., et al., Gene Ther 23: 489-99 and Mutlu, G. M., et al., Am J Respir Crit Care Med 176: 582-90. Briefly, two separate 0.5 ml aliquots of sterile PBS was instilled into mouse lungs for lavaging. The fluid was placed on ice for immediate processing and total number of cells in the lavage was counted using a hemocytometer. Cells from BAL were stained with DIFF-QUIK™ (SIEMENS, Newark, Del.) after cytospin.

Histological Analysis

Lungs were inflated with 20 cc/kg 10% (vol/vol) buffered formalin immediately after mice were killed and used for paraffin-embedded sections. Sections (5 μm) were stained with hematoxylin and eosin, blinded and reviewed for analysis of inflammatory response and pathological changes in the lung.

Pulmonary Permeability Analysis

Pulmonary permeability was measured by Evan's blue dye (EBD) leakage from blood into airways (Baluk, P., et al., Br J Pharmacol 126: 522-8 and Mammoto, A., et al., Nat Commun 4: 1759). Mice (n=7-11) were challenged by intratracheal administration of LPS and, one day later, plasmids expressing α1-ENaC or β1-Na⁺,K⁺-ATPase alone were electroporated to the lungs. EBD (30 mg/kg, SIGMA, St. Louis, Mo.) was administrated by tail-vein injection 47 hrs after gene transfer. One hour later, lungs were perfused with 5 ml of sterile PBS to remove EBD in the vasculature and then removed, photographed, and dried at 60° C. 24 hrs later, EBD was extracted in formamide (FISHER SCIENTIFIC) at 37° C. for 24 hrs and quantified by measuring spectrophotometrically at 620 nm and 740 nm, correcting by using formula E₆₂₀ (EBD)=E₆₂₀−(1.426×E₇₄₀+0.030) (Standiford, T. J., et al., J Immunol 155: 1515-24).

Western Blot Analysis

Western blots were performed as previously described in Lin, X., et al. Am J Respir Crit Care Med 183: 1689-97. Briefly, lung tissues were solubilized in lysis buffer containing protease inhibitor. Thirty μg of total protein was loaded on 10% SDS-PAGE, transferred to PVDF membrane, and probed with primary antibodies against occludin (THERMO FISHER SCIENTIFIC, Waltham, Mass.), ZO-1 (INVITROGEN, Carlsbad, Calif.), or GAPDH (SIGMA-ALDRICH, St. Louis, Mo.). After incubation with secondary antibody and development, bands were detected using the CHEMIDOC Imaging System (BIO-RAD, Hercules, Calif.) and quantified using the IMAGE STUDIO™ LITE software (LI-COR, Lincoln, Nebr.) or the IMAGE LAB software (BIO-RAD, Hercules, Calif.).

Statistical Analysis

Quantitative results are expressed as mean±SEM for in vivo studies and mean±SD for in vitro experiments. The data were evaluated statistically with one way or two way ANOVA and P-values <0.05 were considered statistically significant.

### Results

Gene Transfer of MRCKα to Mice with Pre-Existing LPS-Induced Lung Injury Decreases Multiple Indices of Lung Injury.

Gene transfer of the β1 subunit of the Na⁺,K⁺-ATPase to the lungs of mice can both prevent subsequent LPS-induced lung injury and treat pre-existing LPS-induced lung injury. This reduced pulmonary edema, as measured by graviometric analysis, was due to a combination of both increased active fluid removal from the lung through the function of the Na⁺,K⁺-ATPase and increased barrier function induced by overexpression of the β1 subunit. Since it was shown in cultured cells that the β1 subunit signals through MRCKα to upregulate barrier function, assays were carried out to test whether overexpression of MRCKα could also lead to decreased pulmonary edema in lungs with existing LPS-induced lung injury.

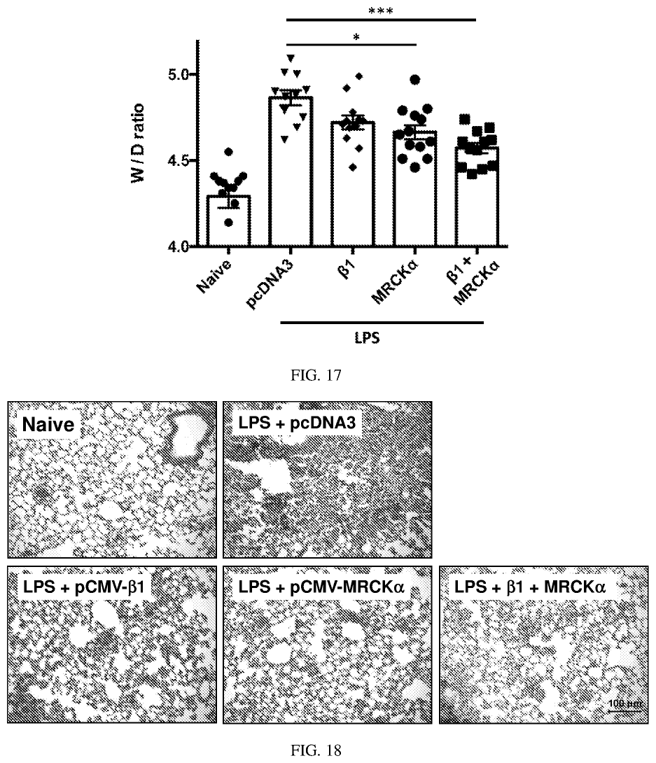

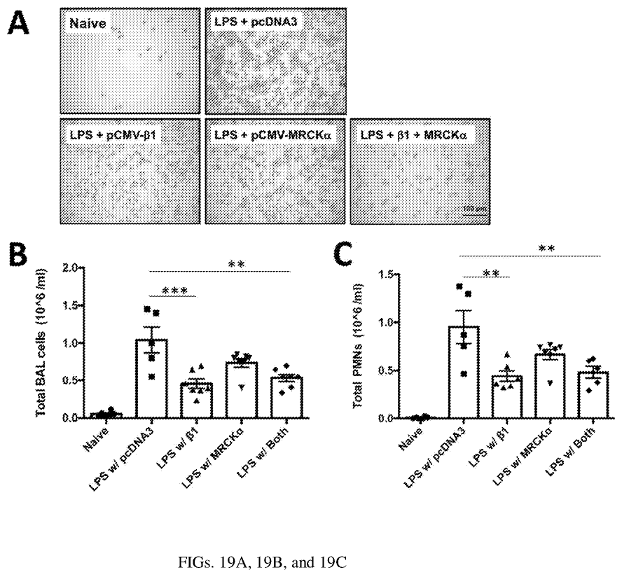

Briefly, mouse lungs were injured by intratracheal administration of LPS (5 mg/kg) and, one day later plasmids expressing β1-Na⁺,K⁺-ATPase or MRCKα were electroporated to the lungs either individually or in combination. Two days later, injury was assessed by measurement of wet-to-dry ratios, histological analysis and BAL protein levels and cellularity. Gene transfer of β1-Na⁺,K⁺-ATPase resulted in reduced wet to dry ratios (FIG. 17), decreased histological signs of injury (FIG. 18), and reduced numbers of total cells and PMNs in the BALF from the mice (FIG. 19), as compared to LPS-injured animals that received a non-expressing, empty control plasmid (pCDNA3). These results confirm previous findings discussed above.

Gene transfer of MRCKα alone reduced the wet-to-dry ratio of animals compared to pcDNA3 to an even slightly greater degree than the β1 subunit of the Na⁺,K⁺-ATPase, and showed reduced lung injury by histology as well as somewhat reduced levels of total cells and PMNs in the BAL, although the decrease did not reach statistical significance (FIGS. 15-19). However, the greatest degree of treatment effect was seen when both β1-Na⁺,K⁺-ATPase or MRCKα plasmids were co-delivered to mice. These results suggest that MRCKα alone can be used to treat existing acute lung injury in mice, but that the greatest effects may be seen in combination with β1-Na⁺,K⁺-ATPase gene transfer as well.

Gene Transfer of MRCKα Alone Increases Levels of Tight Junction Proteins in LPS-Injured Lungs.

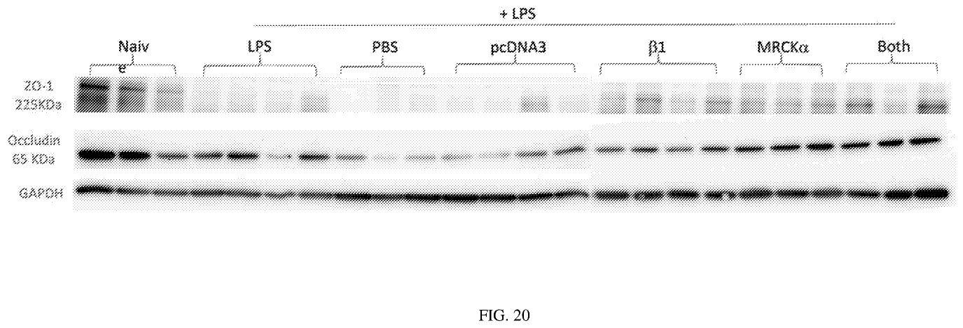

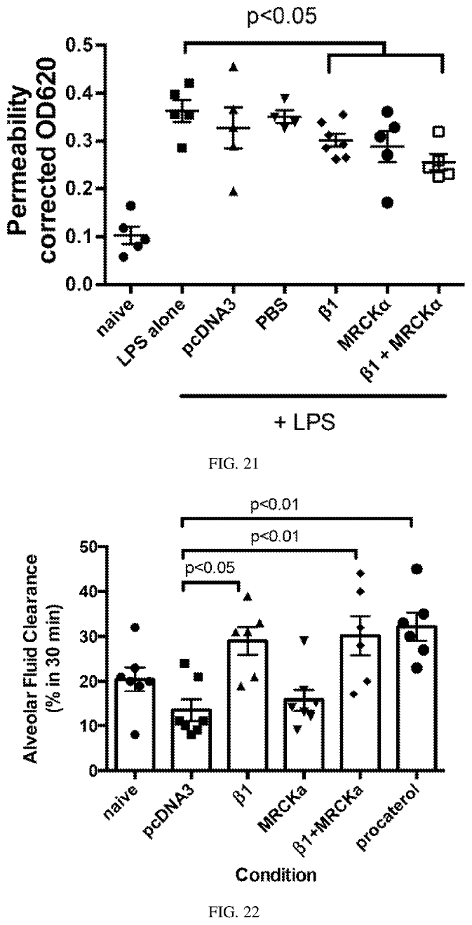

To determine whether MRCKα gene transfer alone could increase levels of tight junction proteins as seen in cultured cells and in the lungs of mice following gene transfer of β1-Na⁺,K⁺-ATPase, expression of tight junction proteins ZO-1 and occludin were measured in both healthy and LPS-injured lungs. Injury of lungs with LPS reduced levels of both ZO-1 and occludin. As shown in FIG. 20, delivery of saline or the control plasmid pcDNA3 had no effect on either ZO-1 or occludin expression in LPS-injured animals. By contrast, delivery of plasmids expressing β1-Na⁺,K⁺-ATPase, MRCKα, or a combination of the two significantly enhanced both ZO-1 and occludin expression in healthy animals by three- to four-fold. Assuming that β1 signals through MRCKα, there was no difference in induction of these tight junction proteins by either plasmid alone or in combination. These results confirm findings in cultured cells that gene transfer of MRCKα alone is sufficient to increase tight junction protein levels at the membrane and tight junction activity.

Gene Transfer of Either the Na⁺,K⁺-ATPase β1 Subunit and/or MRCKα Improves LPS-Injured Lung Permeability in Mice.

To further test whether β1-Na⁺,K⁺-ATPase and MRCKα regulation of tight junctions contributed to its treatment of LPS-induced ALI, lung permeability was measured by Evans Blue dye leakage from blood into airways. Pulmonary leakage in response to LPS was increased three- to four-fold due to endothelial and/or epithelial barrier disruption compared with naïve mice (FIG. 21). Transfer of the control plasmid pcDNA3 after LPS instillation resulted in no change in pulmonary leakage, nor did administration of PBS in the absence of electroporation. Gene transfer of β1-Na⁺,K⁺-ATPase markedly reduced previously LPS-induced pulmonary leakage, compared to LPS alone, as did gene transfer of MRCKα alone or in combination with the β1 subunit. Collectively, these results suggest that gene transfer of MRCKα plays a critical role in inhibiting pulmonary leakage and thus functionally enhances the endothelial and/or epithelial barrier(s).

Overexpression of the Na⁺,K⁺-ATPase β1 Subunit, but not MRCKα Enhances Alveolar Fluid Clearance in Mouse Lungs.

Previous studies have reported that electroporation-mediated gene transfer of β1-Na⁺,K⁺-ATPase increased alveolar fluid clearance (AFC) by 74% in the isolated rat lungs and 43% in mouse lungs. While results in cultured cells point to activation of MRCKα by β1-Na⁺,K⁺-ATPase, assays were carried out to test whether this activation was bi-directional, namely was MRCKα also able to activate the β1-Na⁺,K⁺-ATPase leading to increased AFC.

To determine whether overexpression of MRCKα resulted in increased AFC, plasmids encoding β1-Na⁺,K⁺-ATPase or MRCKα were delivered to mouse lungs by aspiration and electroporation either individually or in combination. Two days later, AFC was measured in live mice using a modification of the mechanically ventilated intact lung model, which maintains ventilation, oxygenation and serum pH (FIG. 22). Mutlu, G. M., et al., Circ Res 94: 1091-100 and Mutlu, G. M., et al., Circ Res 96: 999-1005.

Electroporation of pcDNA3, an empty plasmid, did not increase AFC, compared to naïve mice. By contrast, gene transfer of β1-Na⁺,K⁺-ATPase significantly increased AFC by 115%, higher than that seen previously (Mutlu, G. M., et al., Circ Res 94: 1091-100 and Mutlu, G. M., et al., Circ Res 96: 999-1005). Similarly, the inclusion of procaterol (10⁻⁸ mol/L), the alveolar epithelial β₂-adrenergic receptor specific agonist, in the instillation solution also increased AFC by 145%. However, when MRCKα was overexpressed in mouse lungs following electroporation, there was no increase in AFC over that seen with pcDNA3 or in naïve animals. Further, electroporation of MRCKα in combination with the β1-Na⁺,K⁺-ATPase into mouse lungs failed to increase AFC significantly above that seen with β1-Na⁺,K⁺-ATPase alone. These results suggest that MRCKα does not signal back to increase β1-Na⁺,K⁺-ATPase ion channel activity driving AFC.

Taken together, the results clearly demonstrate that MRCKα overexpression alone in the lungs of mice can treat previously existing LPS-induced acute lung injury by upregulating tight junction protein levels which in turn improve alveolar-capillary epithelial-endothelial barrier function. Following gene delivery of MRCKα alone, pulmonary edema is reduced, histological lung injury is reduced, numbers of infiltrating neutrophils are reduced, and lung permeability is reduced, all without affecting rates or alveolar fluid clearance. Further, when co-administered with the Na⁺,K⁺-ATPase β1 subunit, the effects are even more pronounced. This suggests that MRCKα overexpression may be used as a treatment of ALI/ARDS.

### REFERENCES

1. Rubenfeld G D, Caldwell E, Peabody E, Weaver J, Martin D P, Neff M, et al. Incidence and outcomes of acute lung injury. The New England journal of medicine. 2005; 353(16):1685-93.
2. Villar J, Blanco J, and Kacmarek R M. Current incidence and outcome of the acute respiratory distress syndrome. Curr Opin Crit Care. 2015.
3. Hussain M, Xu C, Ahmad M, Majeed A, Lu M, Wu X, et al. Acute Respiratory Distress Syndrome: Bench-to-Bedside Approaches to Improve Drug Development. Clin Pharmacol Ther. 2018.
4. Bhattacharya J, and Matthay M A. Regulation and repair of the alveolar-capillary barrier in acute lung injury. Annual review of physiology. 2013; 75:593-615.
5. Koval M. Claudin heterogeneity and control of lung tight junctions. Annual review of physiology. 2013; 75:551-67.
6. Overgaard C E, Mitchell L A, and Koval M. In: Fromm M, and Schulzke J D eds. Annals of the New York Academy of Sciences. 2012:167-74.
7. Zihni C, Mills C, Matter K, and Balda M S. Tight junctions: from simple barriers to multifunctional molecular gates. Nature reviews Molecular cell biology. 2016.
8. Peteranderl C, Morales-Nebreda L, Selvakumar B, Lecuona E, Vadasz I, Morty R E, et al. Macrophage-epithelial paracrine crosstalk inhibits lung edema clearance during influenza infection. The Journal of clinical investigation. 2016; 126(4):1566-80.
9. Nieto-Torres J L, Dediego M L, Verdia-Baguena C, Jimenez-Guardeno J M, Regla-Nava J A, Fernandez-Delgado R, et al. Severe acute respiratory syndrome coronavirus envelope protein ion channel activity promotes virus fitness and pathogenesis. PLoS pathogens. 2014; 10(5):e1004077.
10. Helenius I T, Dada L A, and Sznajder J I. Role of ubiquitination in Na,K-ATPase regulation during lung injury. Proceedings of the American Thoracic Society. 2010; 7(1):65-70.
11. Comellas A P, Briva A, Dada L A, Butti M L, Trejo H E, Yshii C, et al. Endothelin-1 impairs alveolar epithelial function via endothelial ETB receptor. American journal of respiratory and critical care medicine. 2009; 179(2):113-22.
12. Factor P, Dumasius V, Saldias F, and Sznajder J I. Adenoviral-mediated overexpression of the NA,K-ATPase beta1 subunit gene increases lung edema clearance and improves survival during acute hyperoxic lung injury in rats. Chest. 1999; 116(1 Suppl):24S-5S.
13. Factor P, Dumasius V, Saldias F, Brown L A, and Sznajder J I. Adenovirus-mediated transfer of an Na+/K+-ATPase beta1 subunit gene improves alveolar fluid clearance and survival in hyperoxic rats. Hum Gene Ther. 2000; 11(16):2231-42.
14. Adir Y, Factor P, Dumasius V, Ridge K M, and Sznajder J I. Na,K-ATPase gene transfer increases liquid clearance during ventilation-induced lung injury. American journal of respiratory and critical care medicine. 2003; 168(12):1445-8.
15. Machado-Aranda D, Adir Y, Young J L, Briva A, Budinger G R, Yeldandi A V, et al. Gene transfer of the Na+,K+-ATPase beta1 subunit using electroporation increases lung liquid clearance. American journal of respiratory and critical care medicine. 2005; 171(3):204-11.
16. Mutlu G M, Machado-Aranda D, Norton J E, Bellmeyer A, Urich D, Zhou R, et al. Electroporation-mediated gene transfer of the Na+,K+-ATPase rescues endotoxin-induced lung injury. American journal of respiratory and critical care medicine. 2007; 176(6):582-90.
17. Emr B M, Roy S, Kollisch-Singule M, Gatto L A, Barravecchia M, Lin X, et al. Electroporation-mediated gene delivery of Na+,K+-ATPase, and ENaC subunits to the lung attenuates acute respiratory distress syndrome in a two-hit porcine model. Shock. 2015;43( 1): 16-23.
18. Lin X, Barravecchia M, Kothari P, Young J L, and Dean D A. betal-Na(+),K(+)-ATPase gene therapy upregulates tight junctions to rescue lipopolysaccharide-induced acute lung injury. Gene therapy. 2016;23(6):489-99.
19. Marconett C N, Zhou B, Sunohara M, Pouldar T M, Wang H, Liu Y, et al. Cross-Species Transcriptome Profiling Identifies New Alveolar Epithelial Type I Cell-Specific Genes. American journal of respiratory cell and molecular biology. 2017;56(3):310-21.
20. Cheek J M, Evans M J, and Crandall E D. Type I cell-like morphotogy in tight alveolar epithelial monolayers. Experimental cell research. 1989;184(2):375-87.
21. Wan H, Winton H L, Soeller C, Stewart G A, Thompson P J, Gruenert D C, et al. Tight junction properties of the immortalized human bronchial epithelial cell lines Calu-3 and 16HBE14o. The European respiratory journal. 2000; 15(6): 1058-68.
22. Hasler U. Wang X, Crambert G. Beguin P. Jaisser F, Horisberger J D, et al. Role of beta-subunit domains in the assembly, stable expression, intracellular routing, and functional properties of Na,K-ATPase. The Journal of biological chemistry. 1998;273(46):30826-35.
23. Paoletli A C, Parmely T J, Tomomori-Sato C, Sato S, Zhu D, Conaway R C, et al. Quantitative proteomic analysis of distinct mammalian Mediator complexes using normalized spectral abundance factors. Proceedings of the National Academy of Sciences of the United States of America. 2006; 103(50): 18928-33.
24. Ashburner M, Ball C A, Blake J A, Botstein D, Butler H, Cherry J M, et al. Gene ontology: tool for the unification of biology. The Gene Ontology Consortium. Nat Genet. 2000;25(1):25-9.
25. The Gene Ontology C. Expansion of the Gene Ontology knowledgebase and resources. Nucleic Acids Res. 2017;45(D1):D331-D8.
26. Campos Y. Qiu X, Gomero E, Wakefield R, Horner L. Brutkowski W, et al. Alix-mediated assembly of the actomyosin-light junction polarity complex preserves epithelial polarity and epithelial barrier. Nat Commun. 2016;7:11876.
27. Leung T, Chen X Q, Tan I, Manser E, and Lim L. Myotonic dystrophy kinase-related Cdc42-binding kinase acts as a Cdc42 effector in promoting cytoskeletal reorganization. Mol Cell Biol. 1998; 18( 1):130-40.
28. Tan I, Seow K T, Lim L, and Leung T. Intermolecular and intramolecular interactions regulate catalytic activity of myotonic dystrophy kinase-related Cdc42-binding kinase alpha. Mol Cell Biol. 2001;21(8):2767-78.
29. Unbekandt M, and Olson M F. The aclin-myosin regulatory MRCK kinases: regulation, biological functions and associations with human cancer. J Mol Med (Berl). 2014;92(3):217-25.
30. Ando K, Fukuhara S, Moriya T, Obara Y, Nakahata N, and Mochizuki N. Rap I potentiates endothelial cell junctions by spatially controlling myosin II activity and actin organization. The Journal of cell biology. 2013 ;202(6):901-16.
31. Gagliardi PA, di Blasio L, Puliafito A, Seano G, Sessa R, Chianale F, et al. PDKI- mediated activation of MRCKalpha regulates directional cell migration and lamellipodia retraction. The Journal of cell biology. 2014;206(3):415-34.
32. Wilkinson S, Paterson HF, and Marshall CJ. Cdc42-MRCK and Rho-ROCK signalling cooperate in myosin phosphorylation and cell invasion. Nature cell biology. 2005;7(3):255-61.
33. Zihni C, Vlassaks E, Terry S, Carlton J, Leung T K C, Olson M, et al. An apical MRCK-driven morphogenetic pathway controls epithelial polarity. Nature cell biology. 2017; 19(9): 1049-60.
34. Marston D J, Higgins C D, Peters K A, Cupp T D, Dickinson D J, Pani A M, et al. MRCK-1 Drives Apical Constriction in C. elegans by Linking Developmental Patterning to Force Generation. Curr Biol. 2016;26(16):2079-89.
35. Unbekandt M, Croft D R, Crighton D, Mezna M, McArthur D, McConnell P, et al. A novel small-molecule MRCK inhibitor blocks cancer cell invasion. Cell Commun Signal. 2014;12:54.
36. Sumi T, Matsumoto K, Shibuya A, and Nakamura T. Activation of LIM kinases by myotonic dystrophy kinase-related Cdc42-binding kinase alpha. The Journal of biological chemistry. 2001 ;276(25):23092-6.
37. Tan I, Yong J, Dong JM, Lim L, and Leung T. A tripartite complex containing MRCK modulates lamellar actomyosin retrograde flow. Cell. 2008; 135(1): 123-36.
38. Lee I C, Leung T, and Tan I. Adaptor protein LRAP25 mediates myotonic dystrophy kinase-related Cdc42-binding kinase (MRCK) regulation of LIMK1 protein in lamellipodial F-actin dynamics. The Journal of biological chemistry. 2014;289(39):26989-7003.
39. Spadaro D, Le S, Laroche T, Mean I, Jond L, Yan J, et al. Tension-Dependent Stretching Activates ZO-1 to Control the Junctional Localization of Its Interactors. Curr Biol. 2017;27(24):3783-95 e8.
40. Shen L, and Turner J R. Actin depolymerization disrupts tight junctions via caveolae-mediated endocytosis. Molecular biology of the cell. 2005; 16(9):3919-36.
41. Nighot P K, and Blikslager A T. Chloride channel CIC-2 modulates light junction barrier function via intracellular trafficking of occludin. American journal of physiology Cell physiology. 2012;302(1):C178-87.
42. Marchiando A M, Shen L, Graham W V, Weber C R, Schwarz B T, Austin J R, 2nd, et al. Caveolin-1-dependent occludin endocytosis is required for TNF-induced tight junction regulation in vivo. The Journal of cell biology. 2010; 189(1):111-26.
43. Smutny M, Cox H L, Leerberg J M, Kovacs E M, Conti M A, Ferguson C, et al. Myosin II isoforms identify distinct functional modules that support integrity of the epithelial zonula adherens. Nature cell biology. 2010; 12(7):696-U147.
44. Itoh M, Tsukita S, Yamazaki Y, and Sugimoto H. Rho GTP exchange factor ARHGEF11 regulates the integrity of epithelial junctions by connecting ZO-l and RhoA-myosin II signaling. Proc Natl Acad Sci USA. 2012;109(25):9905-10.
45. Emr B M, Roy S, Kollisch-Singule M, Gatto L A, Barravecchia M, Lin X, et al. Electroporation Mediated Gene Delivery of Na+,K+-ATPase and ENaC Subunits to the Lung Attenuates Acute Respiratory Distress Syndrome in a Two-Hit Porcine Model. Shock. 2014.
46. Grzesik B A, Vohwinkel C U, Morty R E, Mayer K, Herold S, Seeger W, et al. Efficient gene delivery to primary alveolar epithelial cells by nucleofection. American journal of physiology Lung cellular and molecular physiology. 2013;305(11):L786-94.
47. Torres-Flores J M, and Arias C F. Tight Junctions Go Viral! Viruses. 2015;7(9):5145-54.
48. Hackett T L, de Bruin H G, Shaheen F, van den Berge M, van Oosterhout A J, Postma D S, et al. Caveolin-1 controls airway epithelial barrier function. Implications for asthma. American journal of respiratory cell and molecular biology. 2013;49(4):662-71.
49. Lingrel J B. The Physiological Significance of the Cardiotonic Steroid/Ouabain-Binding Site of the Na,K-ATPase. Annual review of physiology. 2010;72:395-412.
50. Cherniavsky-Lev M. Golani O. Karlish S J, and Garty H. Ouabain-induced internalization and lysosomal degradation of the Na+/K+-ATPase. The Journal of biological chemistry. 2014;289(2): 1049-59.
51. Yoshimura S H, Iwasaka S, Schwarz W, and Takeyasu K. Fast degradation of the auxiliary subunit of Na+/K+-ATPase in the plasma membrane of HeLa cells. J Cell Sci. 2008; 121(Pt 13):2159-68.
52. Flodby P, Kim Y H, Beard L L, Gao D, Ji Y, Kage H, et al. Knockout Mice Reveal a Major Role for Alveolar Epithelial Type I Cells in Alveolar Fluid Clearance. American journal of respiratory cell and molecular biology. 2016.
53. Clifford R J, and Kaplan J H. Regulation of Na,K-ATPase subunit abundance by translational repression. The Journal of biological chemistry, 2009;284(34):22905-15.
54. Zatyka M, Ricketts C, da Silva Xavier G, Minton J, Fenton S, Hofmann-Thiel S, et al. Sodium-potassium ATPase 1 subunit is a molecular partner of Wolframin, an endoplasmic reticulum protein involved in ER stress. Human molecular genetics. 2008; 17(2):190-200.
55. Morton M J, Farr G A, Hull M, Capendeguy O, Horisberger J D, and Caplan M J. Association with {beta}-COP regulates the trafficking of the newly synthesized Na,K-ATPase. The Journal of biological chemistry. 2010;285(44):33737-46.
56. Hatzold J, Beleggia F, Herzig H, Altmuller J, Nurnberg P, Bloch W, et al. Tumor suppression in basal keratinocytes via dual non-cell-autonomous functions of a Na,K-ATPase beta subunit. Elife. 2016;5.
57. Mao H, Ferguson T S, Cibulsky S M, Holmqvisl M, Ding C, Fei H, et al. MONaKA, a novel modulator of the plasma membrane Na,K-ATPase. The Journal of neuroscience: the official journal of the Society for Neuroscience. 2005;25(35):7934-43.
58. Brignone M S, Lanciotti A, Macioce P, Macchia G, Gaetani M, Aloisi F, et al. The betal subunit of the Na,K-ATPase pump interacts with megalencephalic leucoencephalopathy with subcortical cysts protein 1 (MLC1) in brain astrocytes: new insights into MLC pathogenesis. Human molecular genetics. 2011;20(1):90-103.
59. Lanciotti A, Brignone M S, Molinari P, Visentin S, De Nuccio C, Macchia G, et al. Megalencephalic leukoencephalopathy with subcortical cysts protein 1 functionally cooperates with the TRPV4 cation channel to activate the response of astrocytes to osmotic stress: dysregulation by pathological mutations. Human molecular genetics. 2012 ;21(10):2166-80.
60. de Juan-Sanz J, Nunez E, Villarejo-Lopez L, Perez-Hernandez D, Rodriguez-Fraticelli A E, Lopez-Corcuera B, et al. Na+/K+-ATPase is a new interacting partner for the neuronal glycine transporter GlyT2 that downregulates its expression in vitro and in vivo. The Journal of neuroscience: the official journal of the Society for Neuroscience. 2013;33(35):14269-81.
61. Factor P, Saldias F, Ridge K, Dumasius V, Zabner J, Jaffe H A, et al. Augmentation of lung liquid clearance via adenovirus-mediated transfer of a Na,K-ATPase betal subunit gene. The Journal of clinical investigation. 1998; 102(7):1421 -30.
62. Bartolommei G, Moncelli M R, and Tadini-Buoninsegni F. A method to measure hydrolytic activity of adenosinetriphosphatases (ATPases). PloS one. 2013;8(3):e58615.
63. Chruewkamlow N, Pata S, Mahasongkram K, Laopajon W, Kasinrerk W, and Chiampanichayakul S. beta3 subunit of Na,K ATPase regulates T cell activation with no involvement of Na,K ATPase activity. Immunobiology. 2015.
64. Tokhtaeva E, Clifford R J, Kaplan J H, Sachs G, and Vagin O. Subunit Isoform Selectivity in Assembly of Na,K-ATPase alpha-beta Heterodimers. Journal of Biological Chemistry. 2012;287(31 ):26115-25.
65. Hilbers F, Kopec W, Isaksen T J. Holm T H, Lykke-Hartmann K, Nissen P, et al. Tuning of the Na,K-ATPase by the beta subunit. Sci Rep. 2016;6:20442.
66. Pankow S, Bamberger C, Calzolari D, Bamberger A, and Yates J R. 3rd. Deep interactome profiling of membrane proteins by co-interacting protein identification technology. Nature protocols. 2016;11(12):2515-28.
67. Vil O, and Petrak J. Integral membrane proteins in proteomics. How to break open the black box? J Proteomics. 2017; 153:8-20.
68. Gagliardi P A, Blasio L, Puliafito A, Seano G, Chianale F, Sessa R, et al. PDK1 regulates epithelial cell migration through MRCK. Febs J. 2013;280:378.
69. Balasubramaniam S L, Gopalakrishnapillai A, Gangadharan V, Duncan R L, and Barwe S P. Sodium-calcium exchanger 1 regulates epithelial cell migration via calcium-dependent extracellular signal-regulated kinase signaling. The Journal of biological chemistry. 2015;290(20): 12463-73.
70. Acosta-Herrera M, Pino-Yanes M, Perez-Mendez L, Villar J, and Flores C. Assessing the quality of studies supporting genetic susceptibility and outcomes of ARDS. Frontiers in genetics. 2014;5:20.
71. Gagliardi P A, Somale D, Puliafito A, Chiaverina G, di Blasio L, Oneto M, et al. MRCK alpha is activated by caspase cleavage to assemble an apical actin ring for epithelial cell extrusion. Journal of Cell Biology. 2018;217( 1 ):231 -49.
72. Gudipaty S A, and Rosenblatt J. Epithelial cell extrusion: Pathways and pathologies. Semin Cell Dev Biol. 2017;67:132-40.
73. Unbekandl M, Belshaw S, Bower J, Clarke M, Cordes J. Crighton D, el al. Discovery of potent and selective MRCK inhibitors with therapeutic effect on skin cancer. Cancer research. 2018.
74. Dobbs LG, Gonzalez R. and Williams MC. An improved method for isolating type II cells in high yield and purity. The American review of respiratory disease. 1986; 134(1):141 -5.
75. Livak K J, and Schmittgen T D. Analysis of relative gene expression data using real- time quantitative PCR and the 2(T)(-Delta Delta C) method. Methods. 2001;25(4):402-8.
76. Larre I, Lazaro A, Contreras R G, Baida M S, Matter K, Flores-Maldonado C, et al. Ouabain modulates epithelial cell tight junction. Proceedings of the National Academy of Sciences of the United States of America. 2010;107(25):11387-92.
77. Strengert M. and Knaus U G. Analysis of epithelial barrier integrity in polarized lung epithelial cells. Methods in molecular biology. 2011;763:195-206.
78. Tyanova S, Temu T, Sinitcyn P, Carlson A, Hein M Y, Geiger T, et al. The Perseus computational platform for comprehensive analysis of (prote)omics data. Nature methods. 2016; 13(9):731 -40.
79. Morris J H, Knudsen G M, Verschueren E, Johnson J R, Cimermancic P, Greninger A L, et al. Affinity purification-mass spectrometry and network analysis to understand protein-protein interactions. Nature protocols. 2014;9( 11):2539-54.
80. Jorgensen P L, Hakansson K O, and Karlish S J D. Structure and mechanism of Na,K-ATPase: Functional sites and their interactions. Annual review of physiology. 2003;65:817-49.
81. Tokhtaeva E, Sun H, Deiss-Yehiely N, Wen Y, Soni P N, Gabrielli N M, et al. FXYD5 O-glycosylated ectodomain impairs adhesion by disrupting cell-cell trans-dimerization of Na,K-ATPase betal subunits. J Cell Sci. 2016.

The foregoing examples and description of the preferred embodiments should be taken as illustrating, rather than as limiting the present invention as defined by the claims. As will be readily appreciated, numerous variations and combinations of the features set forth above can be utilized without departing from the present invention as set forth in the claims. Such variations are not regarded as a departure from the scope of the invention, and all such variations are intended to be included within the scope of the following claims. All references cited herein are incorporated by reference in their entireties.

1. A method of improving integrity or function of an epithelial or endothelial barrier, comprising increasing a level of myotonic dystrophy kinase-related Cdc42-binding kinases α (MRCKα) in one or more cells in the barrier.

2. The method of claim 1, wherein the epithelial barrier is an alveolar epithelial barrier.

3. The method of claim 1, wherein the level of MRCKα is an enzymatic level or an expression level of MRCKα gene.

4. The method of claim 1, wherein increasing the level of MRCKα comprises introducing an MRCKα polypeptide or a first nucleic acid encoding the MRCKα polypeptide into the one or more cells.

5. The method of claim 1, further comprising increasing a level of Na⁺, K⁺-ATPase (NKA) β1 subunit in the one or more cells.

6. The method of claim 5, wherein the level of NKA β1 subunit is an activity level or an expression level of NKA β1 gene.

7. The method of claim 5, wherein increasing the level of the NKA β1 subunit polypeptide comprises introducing an NKA β1 subunit polypeptide or a second nucleic acid encoding the NKA β1 subunit polypeptide into the cells.

8. The method of claim 1, wherein the cells are alveolar epithelial cells.

9. The method of claim 1, wherein the cells are in vitro.

10. The method of claim 1, wherein the cells are in vivo in a subject.

11. The method of claim 4, wherein the first nucleic acid or the second nucleic acid is in an expression vector.

12. A method of treating a disease or condition associated with compromised function of an epithelial or endothelial barrier comprising increasing a level of MRCKα in one or more cells in the epithelial or endothelial barrier of a subject in need thereof.

13. The method of claim 12, further comprising increasing a level of Na⁺, K⁺-ATPase (NKA) β1 subunit in the one or more cells.

14. The method of claim 12, wherein the disease or condition is selected from the group consisting of acute lung injury, acute respiratory distress syndrome (ARDS), and asthma.

15. A nucleic acid molecule or a set of nucleic acid molecules encoding a MRCKα and/or a NKA β1 subunit.

16. A vector comprising the nucleic acid molecule or the set of nucleic acid molecules of claim 15.

17. A host cell comprising the nucleic acid molecule or the set of nucleic acid molecules of claim 15.

18. A virus or a virus-like particle comprising comprising the nucleic acid molecule or the set of nucleic acid molecules of claim 15.

19. A pharmaceutical composition comprising
(i) the nucleic acid molecule or the set of nucleic acid molecules of claim 15, a vector comprising the nucleic acid molecule or the set of nucleic acid molecules, or a host cell comprising the nucleic acid molecule or the set of nucleic acid molecules, or a virus or a virus-like particle comprising the nucleic acid molecule or the set of nucleic acid molecules and
(ii) a pharmaceutically acceptable carrier or excipient.

20. A kit comprising one or more of the nucleic acid molecule or the set of nucleic acid molecules of claim 15, a vector comprising the nucleic acid molecule or the set of nucleic acid molecules, or a host cell comprising the nucleic acid molecule or the set of nucleic acid molecules, and a virus or a virus-like particle comprising the nucleic acid molecule or the set of nucleic acid molecules.
